(19) United States
(12) Patent Application Publication
(10) Pub. No.: US 2003/0108890 A1
(43) Pub. Date: Jun. 12, 2003

(54) In silico screening for phenotype-associated expressed sequences

(76) Inventors: Anna Vjacheslavovna Baranova, Fairfax, VA (US); Andrey Vladimirovich Lobashev, Moscow (RU); Larisa Leonidovna Krukovskaya, St. Petersburg (RU); Nikolay Kazimirovich Yankovsky, Moscow (RU); Andrey Petrovich Kozlov, St. Petersburg (RU)

Correspondence Address: ROTHWELL, FIGG, ERNST & MANBECK, P.C., 1425 K STREET, N.W., SUITE 800, WASHINGTON, DC, 20005, US

(21) Appl. No.: 10/157,031

(22) Filed: May 30, 2002

Related U.S. Application Data
(60) Provisional application No. 60/293,999, filed on May 30, 2001.
(60) Provisional application No. 60/330,457, filed on Oct. 22, 2001.
(60) Provisional application No. 60/357,144, filed on Feb. 19, 2002.

(51) Int. Cl. C12Q 1/68; G06F 19/00; G01N 33/48; G01N 33/50

(52) U.S. Cl. 435/6; 702/20

(57) ABSTRACT

The present invention provides methods for determining whether a nucleic acid sequence is a marker for a phenotype or cell type of interest which comprises providing a database of expressed sequence tag sequences (EST's) from the species; placing said EST's in groups termed clusters based on homology of EST's within each cluster; determining for each cluster the total number of EST's within said cluster; ordering said clusters sequentially based on the number of EST's in each cluster; dividing said ordered clusters into subranges based on the number of EST's per cluster; determining for each cluster subrange obtained from step (e) the number EST's within said cluster which are expressed in said predetermined cell type of interest; calculating according to a normal distribution the number of clusters in each subrange expected to contain a predetermined threshold percentage of EST's expressed in said cell type of interest, wherein said threshold percentage is a percentage from about 10% to about 100%; determining the number of clusters in each subrange observed to contain said predetermined threshold percentage of EST's expressed in said predetermined cell type; and identifying subranges having an observed number of clusters that meet said predetermined threshold percentage greater than the number of clusters expected to meet said predetermined threshold percentage for the subrange according to normal distribution; wherein if the percentage of EST's expressed in said cell type of interest in a cluster identified is equal to or greater than said predetermined threshold percentage, the cluster contains a nucleic acid that is a marker for the cell type of interest.

The present application is related to, and claims the benefit of priority of, Provisional Application No. 60/293,999, filed May 30, 2001, No. 60/330,457, filed Oct. 22, 2001, and No. 60/357,144, filed Feb. 19, 2002, all of which are incorporated in their entirety by reference herein.

### FIELD OF THE INVENTION

The invention relates generally to the field of genetics and differential expression of genes of interest. More specifically, the invention relates to methods for detecting expression of nucleic acids or proteins associated with a particular phenotype by performing a differential global comparison of a group of Expressed Sequence Tags (EST's) expressed in a particular tissue or cell type with a larger group of available EST's for a plurality of cell types.

The publications and other materials used herein to illuminate the background of the invention or provide additional details respecting the practice are incorporated by reference.

### BACKGROUND OF THE INVENTION

Comparing patterns of gene expression in different cell lines and tissues has important applications for a variety of biological problems. Such information is useful, for example, in comparing mechanisms of differentiation, microbial pathogenesis or tumor malignancy. Typically, such information is obtained by detecting altered gene or protein expression patterns associated with a particular phenotype. Comparing patterns of expression is particularly important, for example, in determining pattern(s) of expression that lead to aberrant cell growth, especially in tumor formation and cancer. A number of experimental methods have been designed for the detection of phenotype or celltype associated gene expression. Most of them are based on time-consuming and expensive experimental protocols (e.g., numerous modifications of the differential display approach, cDNA microarrays, or Serial Analysis of Gene Expression).

EST's are an integral tool in the study of differential expression patterns. The total number of human ESTs in publicly available databases (>4×10⁶) exceeds by approximately two orders of magnitude the total number of different transcripts that can be deduced from the number of human genes (2.5-4×10⁴). Accordingly, there presently exists a need for computer-based procedures for the detection of EST expression profiles to replace traditional experimental protocols utilized in gene expression profiling.

UniGene is an experimental system for automatically partitioning GenBank sequences into a non-redundant set of gene-oriented EST clusters based on DNA sequence homology. Each UniGene cluster contains homologous or similar sequences that represent a unique “gene” or RNA transcript, as well as related information, such as the tissue type(s) in which expression of the transcript has been detected and the map location of the gene encoding the transcript. In addition to sequences of well-characterized genes, hundreds of thousands of novel EST's are also included in the UniGene partitioning system. Clustering is the process of finding subsets of sequences which belong together within a larger set. This is done by converting discrete similarity scores to boolean links between sequences using techniques well known in the art. That is, two sequences are considered linked if their similarity or homology exceeds a threshold. Sequence pairs which are sufficiently similar are linked together to form initial clusters. The set of ESTs is compared with the set of genes using the “megablast” algorithm (Zhang et al., J Comput Biol;7(1-2):203-14 (2000)) and sufficiently similar sequence pairs are added to a particular cluster. A detailed description of clustering performed in the UniGene system can be found at http//www.ncbi.nlm.nih.gov/UniGene.

Differentially expressed EST clusters may be useful as phenotypic markers and prognostic indicators and may be suitable targets for various therapeutic interventions. Prior art methods for the detection of phenotype or cell type of interest or expression patterns have included pairwise comparison of expression patterns in a the phenotype or cell type of interest and corresponding normal tissue in order to determine transcripts which are expressed either specifically or in higher quantities in the cell type of interest. As an example, such pairwise comparisons have been done for tumor-associated expression patterns.

The technique of computer based differential display (CDD) compares expression patterns in a particular tissue versus another tissue source. The comparison can be based on sequence databases available in the World Wide Web. This technique has been used to identify prostate-associated genes (Vasmatzis et al. Proc.Natl. Acad. Sci. USA 95, 300-304 (1998)) or ectopically expressed genes in particular tumor types in comparison to corresponding normal tissue (Schuerle et al. Cancer Res. 60, 40374043 (2000)).

There presently exists a need to develop computer based methods for comparing large numbers of EST's in a global fashion with all known phenotype-associated EST's, so that phenotype-associated patterns of gene expression can be culled from the massive number of such sequences available, without the need for an extensive number of microarray analyses or serial analyses of gene expression in a pairwise manner between a cell type of interest and another individual cell type.

### SUMMARY OF THE INVENTION

The present invention provides methods for the detection of nucleic acid markers associated with a cell type or phenotype of interest by performing a global comparison of a group of EST's known to be expressed in the cell type or phenotype of interest with all EST's expressed in normal tissue in order to identify EST's that are preferentially expressed in the cell or phenotype of interest. The methods comprise arranging both the EST's of interest from a particular species and a larger group of other EST's available for the species in clusters based on homology among the EST's. The methods further comprise arranging the clusters into distinct subranges based on the number of EST's in each cluster and, based on the percentage of EST's derived from the cell type of interest, calculating the number of clusters expected to contain a predetermined percentage of EST's from the cell type of interest. Subranges which contain more than the expected number of clusters containing at least or more than the predetermined percentage of EST's from the cell type are selected for further analysis. The present invention also presents a method for determining a computer based differential display (CDD) of cell or phenotype-associated genes. In one embodiment, the cell or phenotype associated markers are determined for a tumor cell. In a preferred embodiment, at least some of the discrete steps in the method are performed on a computer and comparisons are made between global expression patterns of EST's in a specific cell type or phenotype (such as, e.g, tumor) versus global expression patterns of EST's in all other tissue. Alternatively, the comparisons can be made between EST's expressed in a specific cell type and EST's expressed in normal tissue. The approach was inspired by the hypothesis that evolutionary selective pressures might provide conditions for expression of genes that are not expressed in normal tissue (Kozlov, Medical Hypotheses 46, 81-84 (1996)).

In one embodiment, the invention provides methods for the detection of phenotype or cell type-associated markers by global comparison of all phenotype or cell type-associated EST's with all known EST's to identify EST's that are preferentially expressed in cells expressing the particular phenotype. In a particularly preferred embodiment, the phenotype is tumor formation and the cell type is a tumor cell. Thus, in one embodiment, the invention provides a method for the detection of tumor markers by global comparison of all tumor associated EST's with all known EST's to identify EST's that are preferentially expressed in tumors.

In another embodiment, the invention provides a method for the detection of stress-related genes in a plant model relevant to agricultural plants. Thus, in another preferred embodiment, comparisons are made between global expression patterns of EST's in Arabidopsis thaliana grown in stress conditions (i.e., drought, cold, high salt concentration) versus global expression patterns of EST's in A. thaliana cultivated under normal conditions. Comparisons can also be made between mature plant cells and cells from roots or shoots.

Analysis of combined preparations of mRNAs from several tissues in saturation and experimental subtractive hybridization procedures indicate that tumors contain more diverse sets of mRNAs than any normal tissue. This observation led to the idea of subtracting all available normal EST's (instead of pairwise comparisons) from all available tumor and corresponding normal tissue. (Evtushenko et al. Mol.Biol. 23, 510-520 (1989).

In one embodiment, the invention provides a method for determining whether a nucleic acid sequence is a marker preferentially expressed in a phenotype or cell type of interest from a biological species. In a preferred embodiment, the invention is performed with the aid of statistical software analysis and one or more computers and comprises the following steps: (a) providing a database of expressed sequence tag sequences (EST's); (b) placing said EST's in groups termed clusters based on homology of EST's within each cluster; (c) determining for each cluster the total number of EST's within said cluster; (d) ordering said clusters sequentially based on the number of EST's in each cluster; (e) dividing said ordered clusters into subranges based on the number of EST's per cluster; (f) determining for each cluster subrange obtained from previous step (e) the number EST's within said cluster which are expressed in said predetermined cell type of interest; (g) calculating according to a normal distribution the number of clusters in each subrange expected to contain a predetermined threshold percentage of EST's expressed in said cell type of interest, wherein said threshold percentage is a percentage from about 10% to about 100%; (h) determining the number of clusters in each subrange observed to contain said predetermined threshold percentage of EST's expressed in said predetermined cell type; and (i) identifying subranges having an observed number of clusters that meet said predetermined threshold percentage greater than the number of clusters expected to meet said predetermined threshold for the subrange according to normal distribution; wherein if the percentage of EST's expressed in said cell type of interest in a cluster identified in (i) is equal to or greater than said predetermined threshold percentage, said cluster contains a nucleic acid marker preferentially expressed in the cell type of interest. In preferred embodiments, the clusters of the invention are derived from the UniGene database, which contains all sequences associated with a cluster. The clusters have unique “Hs.” Unigene cluster ID numbers to identify the cluster based on homology. Thus, once a cluster is identified as associated with a phenotype using the EST's from the cluster, the cluster-identifier can be used to identify all other sequences associated with the cluster such as full length mRNA's that are homologous to the EST's in the cluster. In this manner, a reference nucleic acid or polypeptide sequence for the cluster can be determined by reviewing the Unigen database. The methods of the present invention can be used with any database, as long as the database contains sequences that can be arranged in clusters based on homology.

In one embodiment, the invention provides a method for determining whether a nucleic acid is a marker in humans preferentially expressed in a tumor cell. In this embodiment, EST's from a database containing human EST's which contain a description of the source of the EST's retrieved from the cluster description are provided and arranged in individual clusters based on homology; for each cluster the total number of EST's within said cluster is determined; said clusters are ordered sequentially based on the number of EST's in each cluster; said ordered clusters are divided into subranges based on the number of EST's per cluster; the number of EST's within said cluster which are expressed in tumors is determined for each cluster subrange; there is then calculated according to a normal distribution the number of clusters in each subrange expected to contain a predetermined threshold percentage of EST's expressed in tumors, wherein said threshold percentage is a percentage from about 90% to about 100%; the number of clusters is determined in each subrange observed to contain said predetermined threshold percentage of EST's expressed in tumors; and subranges having an observed number of clusters that meet said predetermined threshold percentage greater than the number of clusters expected to meet said predetermined threshold for the subrange according to normal distribution are identified; wherein if the percentage of EST's expressed in said cell type of interest in a cluster from a subrange identified as having a greater than expected number of such clusters is equal to or greater than said predetermined threshold percentage, said cluster contains a nucleic acid marker preferentially expressed in tumors.

In another embodiment, the invention provides a method for detecting EST expression in stress induced A. thaliana which comprises the following steps: (a) for all individual A. thaliana EST clusters, the number of ESTs is retrieved from the cluster description; (b) next, the number of ESTs from all stress-induced cDNA libraries present in each cluster description is counted; (c) there is then determined for each cluster the total number of EST's within said cluster; (d) said clusters are ordered sequentially based on the number of EST's in each cluster; (e) said ordered clusters are then divided into subranges based on the number of EST's per cluster; (f) it is then determined for each cluster subrange obtained from previous step (e) the number of EST's within said cluster which are expressed in Arabidopsis cells presented with stress conditions; (g) there is then calculated according to a normal distribution the number of clusters in each subrange expected to contain a predetermined threshold percentage of EST's expressed in said cell type of interest, wherein said threshold percentage is a percentage from about 10% to about 100%; (h) the number of clusters in each subrange observed to contain said predetermined threshold percentage of EST's expressed in said predetermined cell type is determined; and (i) subranges having an observed number of clusters that meet said predetermined threshold percentage greater than the number of clusters expected to meet said predetermined threshold for the subrange according to normal distribution are identified; wherein if the percentage of EST's expressed in stress-induced plants in a cluster identified in (i) is equal to or greater than said predetermined threshold percentage, said cluster contains a nucleic acid marker preferentially expressed in the stress-induced plants.

The invention thus provides a method for correlating EST expression with a phenotype and in one embodiment requires correlation between a central unit or units containing EST sequence information. In a preferred embodiment, at least some of the EST sequence information analysis is implemented on a conventional personal computer, with the correlator being embodied in a software program. Because the correlator is embodied in software, it may be transported among various computers, which may be used separately or together to perform some or all of the various operations discussed herein.

In another embodiment, the invention provides a method for identifying a tumor cell which comprises detecting the expression of a tumor-associated marker of the present invention. As discussed in greater detail infra, the tumor-associated marker can be a nucleic acid or a polypeptide or fragments thereof.

In another embodiment, the invention provides a method for detecting a tumor cell by detecting the expression of nucleic acid sequences which are tumor-associated and can be used as diagnostic tools for the detection of tumor tissue. The tumor-associated nucleic acids are detected using the methods for determining whether a nucleic acid sequence is a marker for tumors as described herein. The sequences may be utilized for both in vitro and in vivo screening for the presence of a tumor cell. In one embodiment, the invention provides a method for detecting the expression of a tumor-associated nucleic acid sequence wherein the sequence is selected from the group consisting of SEQ ID NO:'s 9, 11, 13, 15, 17, 19, 23, 25, 27, 29, 33, 35, 37, 39, 41, 45, 47, 55, 57, 59, 61, 63, 65, 67, 69, 73, 75, 77, 79, 81, 83, 89, 91, 93, 95, 97, 99, 101, 103, 107, 109, 111, 113, 115, 117, 119, 121, 123, 127, 129, 131, 133, 135, 137, 138, 140, 142, 144, 146, 148, 150, 153, 155, 157, 158, 160, 162, 164, 166, 168, 172, 174, 176, 178, 180, 182, 184, 186, 189, 191, 193, 195, 197, 199, 201, 203, 205, 207, 209, 211, 213, 215, 217, 219, 221, 223, 225, 227, 229, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, and 414. In a particularly preferred embodiment, the nucleic acid sequence is selected from the group consisting of SEQ ID NO:'s 73, 184, 186 and 242.

In another embodiment, the invention provides a method for detecting a tumor cell by detecting the expression of an antigen of a tumor-associated polypeptide which comprises screening tissue or cells with antibodies specific for an antigen expressed by a tumor associated polypeptide, wherein the polypeptide is selected from the group consisting of SEQ ID NO:'s 10, 12, 14, 16, 20, 24, 46, 28, 30, 34, 36, 38, 40, 42, 44, 46, 48, 50, 52, 54, 56, 58, 60, 62, 64, 66, 68, 70, 71, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 139, 141, 143, 145, 147, 149, 151, 152, 154, 156, 159, 161, 163, 165, 167, 169, 171, 173, 175, 177, 179, 181, 183, 185, 187, 188, 190, 192, 194, 196, 198, 200, 202, 204, 206, 208, 210, 212, 214, 216, 218, 220, 222, 224, 226, 228, 231, 233, 235, 237, 239, 241, 243, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 389, 391, 393, 397, 399, 401, 403, 405, 407, 409, 411, 413 and 415. In a preferred embodiment, the invention provides a method for detecting an antigen expressed by a tumor-associated polypeptide selected from the group consisting of SEQ ID NO:'s 74, 185, 187, 188 and 243.

In another embodiment, the invention provides a method for regulating the growth of a tumor cell which comprises regulating the expression of a nucleic acid selected from the group consisting of SEQ ID NO:'s 9, 11, 13, 15, 17, 19, 23, 25, 27, 29, 33, 35, 37, 39, 41, 45, 47, 55, 57, 59, 61, 63, 65, 67, 69, 73, 75, 77, 79, 81, 83, 89, 91, 93, 95, 97, 99, 101, 103, 107, 109, 111, 113, 115, 117, 119, 121, 123, 127, 129, 131, 133, 135, 137, 138, 140, 142, 144, 146, 148, 150, 153, 155, 157, 158, 160, 162, 164, 166, 168, 172, 174, 176, 178, 180, 182, 184, 186, 189, 191, 193, 195, 197, 199, 201, 203, 205, 207, 209, 211, 213, 215, 217, 219, 221, 223, 225, 227, 229, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412 and 414. In a particularly preferred embodiment, the nucleic acid sequence is selected from the group consisting of SEQ ID NO:'s 73, 184, 186 and 242.

In another embodiment, the invention provides a method for regulating the growth of a tumor cell which comprises regulating the expression of a polypeptide selected from the group consisting of SEQ ID NO:'s 10, 12, 14, 16, 20, 24, 46, 28, 30, 34, 36, 38, 40, 42, 44, 46, 48, 50, 52, 54, 56, 58, 60, 62, 64, 66, 68, 70, 71, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 139, 141, 143, 145, 147, 149, 151, 152, 154, 156, 159, 161, 163, 165, 167, 169, 171, 173, 175, 177, 179, 181, 183, 185, 187, 188, 190, 192, 194, 196, 198, 200, 202, 204, 206, 208, 210, 212, 214, 216, 218, 220, 222, 224, 226, 228, 231, 233, 235, 237, 239, 241, 243, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 389, 391, 393, 397, 399, 401, 403, 405, 407, 409, 411, 413 and 415. In a preferred embodiment, the invention provides a method for detecting an antigen expressed by a tumor-associated polypeptide selected from the group consisting of SEQ ID NO:'s 74, 184, 185, 187, 188 and 243.

In another embodiment, the invention provides a method for vaccinating an animal to protect the animal from developing a tumor which comprises administering to the animal an immunogen comprising a polypeptide encoded by a nucleic acid selected from the group consisting of SEQ ID NO:'s 10, 12, 14, 16, 20, 24, 46, 28, 30, 34, 36, 38, 40, 42, 44, 46, 48, 50, 52, 54, 56, 58, 60, 62, 64, 66, 68, 70, 71, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 139, 141, 143, 145, 147, 149, 151, 152, 154, 156, 159, 161, 163, 165, 167, 169, 171, 173, 175, 177, 179, 181, 183, 185, 187, 188, 190, 192, 194, 196, 198, 200, 202, 204, 206, 208, 210, 212, 214, 216, 218, 220, 222, 224, 226, 228, 231, 233, 235, 237, 239, 241, 243, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 389, 391, 393, 397, 399, 401, 403, 405, 407, 409, 411, 413 and 415. In a preferred embodiment, the animal is a human and the immunogen comprises a polypeptide encoded by SEQ ID NO:'s 74, 185, 187, 188 and 243.

### DETAILED DESCRIPTION OF THE INVENTION

In one embodiment, the methods of the present invention can be used to classify data from original dbEST and UNIGENE databases in a table form (Baranova et al., FEBS Letters, 508, 143-148 (2001)). The HSAnalyst program is one type of software program that can be used to assemble the EST sequences and clusters using the methods of the present invention. This program is available at (http//pcn197.vigg.ru/programs/HSAnalyst.exe). In one preferred embodiment, the methods of the invention comprise the compiling of a supplemental database which contains only those sets of EST's that can specifically be associated with expression in either a particular abnormal (e.g., tumor)or normal physiological condition or tissue type. In one embodiment, the supplemental database includes EST entries from all human cDNA libraries that can specifically be classified as <<tumor>> or <<normal>> by tissue source. The supplemental database utilized in the demonstrative examples of the present invention contains a carefully checked description of each included library, cross-referenced from different data sources such as dbEST, UNIGENE and CGAP web-sites, which are available at the National Institutes of Health web site (www.ncbi.nlm.nih.gov), TIGR (www.tigr.org) and Stratagene (www.stratagene.com). The supplemental database thus contains a classification of all cDNA libraries as either tumor or normal. Approximately 4000 entries in the supplemental database describing cDNA sources were classified according to their origin from tumor or normal tissues (cells). In checking the libraries, those obtained from “premalignant”, “non-cancerous pathology” and “immortalized cells” were not included in the supplemental database. In other embodiments, one or more databases can be utilized in the methods of the invention without modifying in a supplemental database. In the case of the databases used in the demonstrative examples presented herein, some of the libraries were considered undefined due to lack of information or ambiguity of information.

EST pre-classification in the supplemental databases for other possible tasks not described herein can be performed by users themselves

HSAnalyst software was able to arrange EST data in the supplemental database according to any given parameter, e.g. tissue type or the number of ESTs contained in a cluster. As will readily be appreciated by persons of ordinary skill in the art, classification of ESTs according to tissue types requires verification of available database information on expression patterns and is the most time-consuming stage. Depending on the type of tissue being analyzed for global expression patterns, a specific database may contain and compare only sequences that are conclusively known to be expressed in a given cell type or physiological state. Classification of the data can be performed by many variations of software capable of handling large groups of data from the UniGene database without deviating from the scope of the present invention.

In one embodiment, the present invention provides a method for the detection of tumor markers wherein the CDD approach is utilized to search various publicly available databases containing human EST's. This gene-hunting procedure was inspired by the hypothesis that tumors may provide conditions for the expression of some transcribed units that are not expressed in any normal tissues. Instead of pairwise comparison of each tumor and corresponding normal tissue, a differential display of all available tumor libraries against all available normal libraries was performed.

A particular feature of the methods of the present invention includes subtracting all available clusters containing more than 10% of normal-derived ESTS from a whole set of the UniGene clusters to identify clusters associated with a particular phenotype, instead of pairwise comparisons of each tumor and corresponding normal tissue.

EST's present a particularly useful set of sequence data to analyze with the methods of the present invention. GenBank included 3,900,480 human ESTs as of Nov. 16, 2001. These sequences and the methods of the present invention were used to generate Table 1 discussed infra. UniGene includes all human ESTs clustered by homology. It should be noted that as available sequence data on EST's continues to grow, these numbers correspondingly change. The methods of the present invention will be equally applicable, however, to the evolving database resources which continue to become available for sequence analysis.

Most EST's can be traced to a certain tissue source, including tumor and normal ones. In a particularly preferred embodiment, the comparison of tumor and normal libraries is performed on a supplemental database referred to herein as “LibraryRegistry”, which comprises a supplemental database that contains only those EST's that clearly are defined as originally detected in normal or tumor tissue samples, as discussed above. It can readily be appreciated by persons of ordinary skill in the art that similar methods can be employed to “customize” a database to include only sequences known to be associated with a particular phenotype or cell type and a defined set of “normal” sources which provide sequences that can be distinguished from the cell or phenotype of interest. Just as the present invention provides tumor-associated EST's and compares these to other human EST's, an example is also provided which compares EST's reported from stress-induced Arabidopsis and EST's from Arabidopsis that are not from plants exposed to the stress conditions.

A preferred embodiment of the invention utilizes a method of sequence comparison to determine tumor-associated EST's. This method is demonstrated on tumor-specific sequences but as noted is applicable to any well-described database which provides information on the origin of nucleic acid sequences contained therein. In the first step, a database of clustered EST sequences containing a description of the source for each of the sequences is selected for analysis. In the second step, for each cluster the number of its ESTs is retrieved from the cluster description. Next, the number of ESTs from the “tumor” cDNA libraries is counted. The whole range of possible EST numbers is dissected into sub ranges. The arrangement of sub ranges can be performed exponentially (e.g., sub ranges with exponents 1-2, 34, 5-8,9-16) or linearly (sub ranges with factors 1-10, 11-20, 21-30). Simultaneously, the tumor ESTs/all ESTs percentage is calculated for each cluster and those clusters which exceed a user-defined bottom threshold value for the percentage of tumor ESTs/all ESTs are listed in the output file as tumor specific clusters.

The subranges can be arranged exponentially (e.g., sub ranges with exponents 1-2,3-4, 5-8,9-16) or linearly (sub ranges e.g. with factors 1-10, 11-20, 21-30). Classification of subranges into linear or logarithmic format provides two complementary ways for statistical estimation of a threshold level for determining whether a cluster is associated with a particular phenotype. Using the methods of the present invention, arrangement of subranges produced successful detection of tumor-associated markers whether subranges were arranged linearly as in Table 1 or logarithmically. Program output is designed to separate information about each set of clusters of the same size. In general it is possible to choose some intervals within the whole range of cluster sizes (cluster “size” is the number of EST's in a cluster). For example, if one needs the detailed picture of tumor clusters distribution it may be useful to choose narrow intervals, even assigning a cluster to as little as 1 EST sequence. For each interval the following values are calculated: total number of ESTs contained in clusters of the size within the interval N_{EST}, total number of these clusters N_{clust} and the number of tumor related clusters Nₜᵤₘ within this interval. Tumor related clusters that have relative content of tumor tissue-derived ESTs over the threshold denoted as <<t>> given by user (usually from 90% to 100%). Also, the theoretically expected number of tumor clusters within this interval is calculated. To let a computer program do this, the user must input the expected contents of tumor-related ESTs in the whole database. Given the N_{EST} and N_{clust} for the interval it is assumed that tumor cluster distribution is binomial so the expected number of tumor clusters is Nₜᵤₘ=N_{clust}*ΣCₘp^{m}(1−p)^{n-m} where p is mean tumor ESTs content in database (declared by user). The sum in the brackets is calculated for each m: n*t<m<n, where n varies between the interval edges and represents the hypothetical cluster size. The 90-100% threshold range described above for cell type-associated clusters in humans is for the case of human tumor-associated EST's but this number can vary depending on the difference between the expected number of clusters at a given t for a cluster size versus the observed number of clusters at a given t for the cluster size.

In an exemplary analysis using the methods of the present invention, the database LibraryRegistry was analyzed. This library provided a database of EST's from human normal and tumor sources. The EST's were placed in clusters based on homology; for each cluster the total number of EST's within the cluster was determined, the clusters were then ordered sequentially based on the number of EST's in each cluster and divided into subranges linearly based on the number of EST's per cluster as shown in Table 1. For each cluster subrange obtained the number EST's within said cluster expressed in tumor cells was determined. Next, based on a normal distribution, the number of clusters in each subrange expected to contain a predetermined threshold percentage of EST's expressed in tumor cells was calculated, wherein the threshold percentage was calculated at 90% and 100%. The number of clusters in each subrange observed to contain 90% or 100% tumor-specific EST's was determined. Next, subranges having an observed number of clusters that meet said predetermined threshold percentage five times greater than the number of clusters expected to meet said predetermined threshold for the subrange according to normal distribution were noted. Clusters in the subranges between 17 and 2048 were determined to contain 5 times or greater the number of expected clusters having 90% or more tumor-derived EST's in the cluster subrange were identified. These clusters were than associated with the corresponding Hs. Identifying number from the Unigene database to determine the nucleic acid sequences which were tumor-associated sequences.

To be sure that what was found was a “true” tumor-associated cluster not generated by chance among the total number of EST clusters classified with the methods of the present invention, the theoretical number of “tumor” clusters for every sub range is calculated. This is done utilizing an underlying model of a unimodal binomial distribution with the mean value of “tumor/all” percentage that can be defined by the user (0 to 100%). This binomial method is used to determine the expected number of tumor/all for predetermined thresholds for each cluster size based on the proportion of EST's from tumor cells in the database. In the example described in Table 1, the subranges which were analyzed for 90% or more tumor derived EST's were subranges that contained at least five times more such clusters than expected for the cluster size. This ratio of observed to expected has been found by the inventors to be reliable for determining phenotype or cell type associated clusters utilizing databases from Arabidopsis, human and mouse. It will readily be appreciated by persons of ordinary skill in the art that other ratios of observed/expected clusters for a predetermined threshold will also be useful. As little as 3.5 times the number of observed/expected clusters equal to or greater than the threshold range are also contemplated. Clusters between 3.5 and 5 times the number of expected clusters may also identify useful subranges displaying the predetermined threshold percentage of sequences for a cluster. Alternatively, an observed number of clusters for subrange that is at least one standard deviation greater than the number of clusters expected for a subrange may also be used to identify useful subranges displaying the predetermined threshold percentage of sequences for a cluster.

Referring now to Table I, the expected numbers of tumor-specific clusters that exceeded threshold values were calculated for a UniGene database of human EST's that was available on Nov. 6, 2001. A comparison between the expected and observed tumor-derived EST's demonstrated that tumor-related clusters were not accidental but represented a natural phenomenon. In this example, user-derived threshold values for the percentage of tumor-derived EST's to all EST's were at least 90% tumor-derived EST's per cluster and 100% tumor-derived EST's per cluster. When at least 90% of the EST's in a cluster are tumor derived, the cluster is referred to as tumor-associated. Each cluster was identified with a representative nucleic acid sequence based on the Hs. number for the sequence and the representative longest nucleotide sequence or defined mRNA sequence associated with the cluster.

Referring now to Table II, there are shown the results of tumor-related clusters detected with the methods of the present invention on a Unigene database that was assembled May 3, 2002. Except for the methods otherwise noted, the methods used to determine markers for tumors were as described for Table II. All of the tumor associated clusters in Table II had a number of EST's per cluster of 10 or more, which was found to be a significant number of EST's that would be tumor-associated using the methods described herein for identifying subranges having an observed number of clusters that was five times more than the expected number of clusters that met a predetermined threshold of 90% or more tumor derived sequences. Among the 196 tumor related clusters detected, 93 are non-coding and 103 encode at least one polypeptide sequence. Among clusters encoding a polypeptide, six correspond to known genes previously described as tumor markers/antigens, as indicated in Table 2.

Differentially expressed EST clusters are useful as markers for a physiological state or phenotype and prognostic indicators and may be suitable targets for various therapeutic interventions. Therapeutic interventions can include use of various gene therapy techniques to regulate the expression of the sequences, target-associated antibodies to inhibit growth of cells expressing phenotype associated marker polypeptides, and use of marker polypeptides as immunogens to vaccinate an animal against cells expressing the marker.

Useful diagnostic techniques include, but are not limited to fluorescent in situ hybridization (FISH), direct DNA sequencing, PFGE analysis, Southern blot analysis, single stranded conformation analysis (SSCA), RNase protection assay, allele-specific oligonucleotide (ASO), dot blot analysis and PCR-SSCP, as discussed in detail further below. Also useful is the recently developed technique of DNA microchip technology.

“Antibodies.” The present invention also provides polyclonal and/or monoclonal antibodies and fragments thereof, and immunologic binding equivalents thereof, which are capable of specifically binding to the tumor-associated polypeptides and fragments thereof or to polynucleotide sequences from the tumor-associated region, particularly from the tumor-associated locus or a portion thereof. The term “antibody” is used both to refer to a homogeneous molecular entity, or a mixture such as a serum product made up of a plurality of different molecular entities. Antibodies to the tumor-associated markers will be useful in assays as well as pharmaceuticals.

As used herein, the term “computer” is meant to refer to at least one computer but can also include more than one computer connected by any means known in the art of computer science. Furthermore, the term is also meant to include a computer interacting with a remote computer or other server which provides access to a plurality of databases via the world wide web. In one embodiment, the analysis of EST clusters is performed on software on a computer, while the information imported to the computer for correlation is obtained from contact with the world wide web.

Alteration of mRNA expression for the tumor markers of the present invention can be detected by any techniques known in the art. These include Northern blot analysis, PCR amplification and RNase protection. Alteration of expression of tumor-associated genes can also be detected by screening for alteration of the expression of the protein encoded by a tumor-associated gene. For example, monoclonal antibodies immunoreactive with a marker polypeptide can be used to screen a tissue using methods known in the art. These include Western blots, immunohistochemical assays and ELISA assays. Functional assays, such as protein binding determinations, can be used and assays biochemical function of a tumor-associated marker can be employed.

Genes or gene products can also be detected in human body samples, such as serum, stool, urine and sputum and isolated tumor tissue. The same techniques discussed above for detection of genes or gene products in tissues can be applied to other body samples. Cancer cells are sloughed off from tumors and appear in such body samples. In addition, the gene product itself may be secreted into the extracellular space and found in these body samples even in the absence of cancer cells. By screening such body samples, a simple early diagnosis can be achieved for many types of cancers. In addition, the progress of chemotherapy or radiotherapy can be monitored more easily by testing such body samples for genes or gene products. The diagnostic methods of the present invention is useful for clinicians, so they can decide upon an appropriate course of treatment.

Pairs of single-stranded DNA primers can be annealed to sequences within or surrounding a tumor-associated gene in order to prime amplifying DNA synthesis of the gene itself. A complete set of these primers allows synthesis of all of the nucleotides of the gene coding sequences, i.e., the exons. The set of primers preferably allows synthesis of both intron and exon sequences. The primers themselves can be synthesized using techniques which are well known in the art. Generally, the primers can be made using oligonucleotide synthesizing machines which are commercially available. Given the sequences of the tumor associated genes of the invention, design of particular primers is well within the skill of the art.

The nucleic acid probes provided by the present invention are useful for a number of purposes. They can be used as probes to detect PCR amplification products derived from the mRNA of the gene or to detect actual mRNA transcripts directly in tumors or other cells being analyzed for expression of tumor-associated markers.

“Probes”. Polynucleotide probes form a stable hybrid with a of the target sequence, under highly stringent to moderately stringent hybridization and wash conditions. If it is expected that the probes will be perfectly complementary to the target sequence, high stringency conditions will be used. Hybridization stringency may be lessened if some mismatching is expected, for example, if variants are expected with the result that the probe will not be completely complementary. Conditions are chosen which rule out nonspecific/adventitious bindings, that is, which minimize noise. In general, hybridizations conditions will be stringent conditions.

Probes for the tumor-associated markers may be derived from the sequences of the region or its cDNAs. The probes may be of any suitable length, which span all or a portion of the marker, and which allow specific hybridization to the transcripts expressed from the marker. If the target sequence contains a sequence identical to that of the probe, the probes may be short, e.g., in the range of about 8-30 base pairs, since the hybrid will be relatively stable under even highly stringent conditions. If some degree of mismatch is expected with the probe, i.e., if it is suspected that the probe will hybridize to a variant region, a longer probe may be employed which hybridizes to the target sequence with the requisite specificity.

The probes may include an isolated polynucleotide attached to a label or reporter molecule and may be used to isolate other polynucleotide sequences, having sequence similarity by standard methods. Other similar polynucleotides may be selected by using homologous polynucleotides. Alternatively, polynucleotides encoding these or similar polypeptides may be synthesized or selected by use of the redundancy in the genetic code. Various codon substitutions may be introduced, e.g., by silent changes (thereby producing various restriction sites) or to optimize expression for a particular system.

Probes comprising synthetic oligonucleotides or other polynucleotides of the present invention may be derived from naturally occurring or recombinant single- or double-stranded polynucleotides, or be chemically synthesized. Probes may also be labeled by nick translation, Klenow fill-in reaction, or other methods known in the art.

Portions of the polynucleotide sequence having at least about eight nucleotides, usually at least about 15 nucleotides, and fewer than about 6 kb, usually fewer than about 1.0 kb, from a polynucleotide sequence encoding the tumor associated markers of the invention are preferred as probes. Thus, this definition includes probes of 8, 12, 15, 20, 25, 40, 60, 80, 100, 200, 300, 400 or 500 nucleotides or probes having any number of nucleotides within these ranges of values (e.g., 9, 10, 11, 16, 23, 30, 38, 50, 72, 121, etc., nucleotides), or probes having more than 500 nucleotides. The probes may also be used to determine whether mRNA encoding a tumor-associated marker is present in a cell or tissue. The present invention contemplates the use of probes having at least 8 nucleotides derived from a tumor-associated marker of the invention and any combination of these sequences as described in further detail below, its complement or functionally equivalent nucleic acid sequences.

Similar considerations and nucleotide lengths are also applicable to primers which may be used for the amplification of all or part of the tumor-associated markers of the invention. Thus, a definition for primers includes primers of 8, 12, 15, 20, 25, 40, 60, 80, 100, 200, 300, 400, 500 nucleotides, or primers having any number of nucleotides within these ranges of values (e.g., 9, 10, 11, 16, 23, 30, 38, 50, 72, 121, etc. nucleotides), or primers having more than 500 nucleotides, or any number of nucleotides between 500 and 9000. The primers may also be used to determine whether mRNA encoding a tumor-associated marker is present in a cell or tissue.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter.

Probe sequences may also hybridize specifically to duplex DNA under certain conditions to form triplex or other higher order DNA complexes. The preparation of such probes and suitable hybridization conditions are well known in the art.

Methods of Use: Nucleic Acid Diagnosis and Diagnostic Kits

In order to detect the presence of neoplasia, the progression toward malignancy of a precursor lesion, or as a prognostic indicator, a biological sample of the lesion is prepared and analyzed for the presence or absence of the expression of a tumor-associated marker. Results of these tests and interpretive information are returned to the health care provider for communication to the tested individual. Such diagnoses may be performed by diagnostic laboratories, or, alternatively, diagnostic kits are manufactured and sold to health care providers or to private individuals for self-diagnosis.

Initially, the screening method may involve amplification of the relevant sequences. In another preferred embodiment of the invention, the screening method involves a non-PCR based strategy. Both PCR and non-PCR based screening strategies can detect target sequences with a high level of sensitivity.

The most popular method used today is target amplification. Here, the target nucleic acid sequence is amplified with polymerases. One particularly preferred method using polymerase-driven amplification is the polymerase chain reaction (PCR). The polymerase chain reaction and other polymerase-driven amplification assays can achieve over a million-fold increase in copy number through the use of polymerase-driven amplification cycles. Once amplified, the resulting nucleic acid can be sequenced or used as a substrate for DNA probes.

When the probes are used to detect the presence of the target sequences, the biological sample to be analyzed, such as blood or serum, may be treated, if desired, to extract the nucleic acids. The sample nucleic acid may be prepared in various ways to facilitate detection of the target sequence; e.g. denaturation, restriction digestion, electrophoresis or dot blotting. The targeted region of the analyte nucleic acid usually must be at least partially single-stranded to form hybrids with the targeting sequence of the probe. If the sequence is naturally single-stranded, denaturation will not be required. However, if the sequence is double-stranded, the sequence will probably need to be denatured. Denaturation can be carried out by various techniques known in the art.

Analyte nucleic acid and probe are incubated under conditions which promote stable hybrid formation of the target sequence in the probe with the putative targeted sequence in the analyte. The region of the probes which is used to bind to the analyte can be made completely complementary to a targeted region. Therefore, high stringency conditions are desirable in order to prevent false positives. However, conditions of high stringency are used only if the probes are complementary to regions of the chromosome which are unique in the genome. The stringency of hybridization is determined by a number of factors during hybridization and during the washing procedure, including temperature, ionic strength, base composition, probe length, and concentration of formamide. Under certain circumstances, the formation of higher order hybrids, such as triplexes, quadraplexes, etc., may be desired to provide the means of binding target sequences.

Detection, if any, of the resulting hybrid is usually accomplished by the use of labeled probes. Alternatively, the probe may be unlabeled, but may be detectable by specific binding with a ligand which is labeled, either directly or indirectly. Suitable labels, and methods for labeling probes and ligands are known in the art, and include, for example, radioactive labels which may be incorporated by known methods (e.g., nick translation, random priming or kinasing), biotin, fluorescent groups, chemiluminescent groups (e.g., dioxetanes, particularly triggered dioxetanes), enzymes, antibodies and the like. Variations of this basic scheme are known in the art, and include those variations that facilitate separation of the hybrids to be detected from extraneous materials and/or that amplify the signal from the labeled moiety. A number of these variations are reviewed in e.g., U.S. Pat. No. 4,868,105, and in EPO Publication No. 225,807.

Once a sufficient quantity of desired tumor-associated polypeptide has been obtained, it may be used for various purposes. A typical use is the production of antibodies specific for binding. These antibodies may be either polyclonal or monoclonal, and may be produced by in vitro or in vivo techniques well known in the art. For production of polyclonal antibodies, an appropriate target immune system, typically mouse or rabbit, is selected. Substantially purified antigen is presented to the immune system in a fashion determined by methods appropriate for the animal and by other parameters well known to immunologists. Typical sites for injection are in footpads, intramuscularly, intraperitoneally, or intradermally. Of course, other species may be substituted for mouse or rabbit. Polyclonal antibodies are then purified using techniques known in the art, adjusted for the desired specificity.

An immunological response is usually assayed with an immunoassay. Normally, such immunoassays involve some purification of a source of antigen, for example, that produced by the same cells and in the same fashion as the antigen. A variety of immunoassay methods are well known in the art.

Monoclonal antibodies with affinities of 10-8 M-1 or preferably 10-9 to 10-10 M-1 or stronger will typically be made by standard procedures. Briefly, appropriate animals will be selected and the desired immunization protocol followed. After the appropriate period of time, the spleens of such animals are excised and individual spleen cells fused, typically, to immortalized myeloma cells under appropriate selection conditions. Thereafter, the cells are clonally separated and the supernatants of each clone tested for their production of an appropriate antibody specific for the desired region of the antigen.

Other suitable techniques involve in vitro exposure of lymphocytes to the antigenic polypeptides, or alternatively, to selection of libraries of antibodies in phage or similar vectors. The polypeptides and antibodies of the present invention may be used with or without modification. Frequently, polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent agents, chemiluminescent agents, magnetic particles and the like. Patents teaching the use of such labels include U.S. Pat. Nos. 3,817,837; 3,850,752; 3,939,350; 3,996,345; 4,277,437; 4,275,149 and 4,366,241. Also, recombinant immunoglobulins may be produced (see U.S. Pat. No. 4,816,567).

Methods of Use: Peptide Diagnosis and Diagnostic Kits

Antibodies (polyclonal or monoclonal) may be used to detect the absence or absence of peptides encoded by tumor-associated markers of the invention. Techniques for raising and purifying antibodies are well known in the art and any such techniques may be chosen to achieve the preparations claimed in this invention. In a preferred embodiment of the invention, antibodies will immunoprecipitate proteins from solution as well as react with proteins on Western or immunoblots of polyacrylamide gels. In another preferred embodiment, antibodies will detect tumor-associated proteins in paraffin or frozen tissue sections, using immunocytochemical techniques. Antibodies specific to tumor-associated markers described herein can be employed in conjunction with toxic products that can be bound to the antibodies and selectively delivered to tumor cells via binding of the antibody with the tumor-associated polypeptide present on or in the tumor cell utilizing techniques well known in the art.

Preferred embodiments relating to methods for detecting tumor-associated proteins include enzyme linked immunosorbent assays (ELISA), radioimmunoassays (RIA), immunoradiometric assays (IRMA) and immunoenzymatic assays (IEMA), including sandwich assays using monoclonal and/or polyclonal antibodies. Exemplary sandwich assays are described by David et al. in U.S. Pat. Nos. 4,376,110 and 4,486,530.

Methods of Use: Antisensense and siRNA Therapy

The present invention contemplates an antisense polynucleotide up to about 50 nucleotides in length that hybridizes with mRNA molecules that encode a tumor-associated polypeptide, and the use of one or more of those polynucleotides in treating cancer cells. See U.S. Pat. Nos. 5,891,858 and 5,885,970, incorporated herein by reference, for further details. The antisense polynucleotide or siRNA is useful for treating cancer caused by expression of a tumor-specific or tumor-associated polypeptide. In a similar manner, siRNA molecules specific for tumor-associated nucleic acid markers of the invention can also be used to suppress transcription of said marker sequences.

In one embodiment an antisense polynucleotide or siRNA is contacted with a cancer cell. The contact is carried out in vivo in a host animal, and contact is effected by administration to the animal of a pharmaceutical composition containing the polynucleotide dissolved or dispersed in a physiologically tolerable diluent so that a body fluid such as blood or lymph provides at least a portion of the aqueous medium. In vivo contact is maintained until the polynucleotide is eliminated from the mammal's body by a normal bodily function such as excretion in the urine or feces or enzymatic breakdown. The polynucleotide may be injected directly into the tumor in an aqueous medium (an aqueous composition) via a needle or other injecting means and the composition is injected throughout the tumor as compared to being injected in a bolus. For example, an aqueous composition containing an antisense polynucleotide or siRNA, the inverts or mixtures thereof is injected into tumors via a needle. The needle is placed in the tumors and withdrawn while expressing the aqueous composition within the tumor. That mode of administration is carried out in three approximately orthogonal planes in the tumors.

This administration technique has the advantages of delivering the polynucleotide directly to the site of action and avoids most of the usual body mechanisms for clearing drugs. Tumors can be located using e.g., modern imaging techniques such as X-ray, ultrasound and MRI so that exact placement of the polynucleotide can be carried out.

A polynucleotide can also be administered in the form of liposomes. As is shown in the art, liposomes are generally derived from phospholipids or other lipid substances. Liposomes are formed by mono or multi-lamellar hydrated liquid crystals that are dispersed in an aqueous medium. Any non-toxic, physiologically acceptable and metabolizable lipid capable of forming liposomes can be used. The present compositions in liposome form can contain stabilizers, preservatives, excipients, and the like in addition to the agent.

An antisense polynucleotide or siRNA can also be administered by gene therapy. The polynucleotide may be introduced into the cell in a vector such that the polynucleotide remains extrachromosomal. In such a situation, the polynucleotide will be expressed by the cell from the extrachromosomal location. Vectors for introduction of polynucleotides for extrachromosomal maintenance are known in the art, and any suitable vector may be used. Methods for introducing DNA into cells such as electroporation, calcium phosphate coprecipitation and viral transduction are known in the art, and the choice of method is within the competence of a person of ordinary skill in the art.

The antisense polynucleotide or siRNA, may be employed in gene therapy methods in order to decrease the amount of the expression products in cancer cells, especially in those cases where overexpressed. Such gene therapy is particularly appropriate for use in both cancerous and pre-cancerous cells.

Gene therapy would be carried out according to generally accepted methods, for example, as described in further detail in U.S. Pat. No. 5,747,282 and references cited therein, all incorporated by reference herein. Expression vectors in the context of gene therapy are meant to include those constructs containing sequences sufficient to express a polynucleotide that has been cloned therein. In viral expression vectors, the construct contains viral sequences sufficient to support packaging of the construct. If the polynucleotide encodes an antisense polynucleotide or siRNA or a ribozyme, expression will produce the antisense polynucleotide or siRNA or ribozyme. Thus in this context, expression does not require that a protein product be synthesized. In addition to the polynucleotide cloned into the expression vector, the vector also contains a promoter functional in eukaryotic cells. The cloned polynucleotide sequence is under control of this promoter. Suitable eukaryotic promoters include those described above. The expression vector may also include sequences, such as selectable markers and other sequences conventionally used.

Gene transfer techniques which target DNA directly to specific tumor cell types are preferred. Receptor-mediated gene transfer, for example, is accomplished by the conjugation of DNA (usually in the form of covalently closed supercoiled plasmid) to a protein ligand via polylysine. Ligands are chosen on the basis of the presence of the corresponding ligand receptors on the cell surface of the target cell/tissue type. These ligand-DNA conjugates can be injected directly into the blood if desired and are directed to the target tissue where receptor binding and internalization of the DNA-protein complex occurs. To overcome the problem of intracellular destruction of DNA, coinfection with adenovirus can be included to disrupt endosome function.

Methods of Use: Transformed Hosts; Transgenic/Knockout Animals and Models

In one embodiment of the invention, a transgene is introduced into a non-human host to produce a transgenic animal expressing a human or murine tumor-specific or tumor-associated gene. The transgenic animal is produced by the integration of the transgene into the genome in a manner that permits the expression of the transgene. Methods for producing transgenic animals are generally described e.g., in U.S. Pat. No. 4,873,191.

Transgenic animals may be produced from the fertilized eggs from a number of animals including, but not limited to reptiles, amphibians, birds, mammals, and fish. Within a particularly preferred embodiment, transgenic mice are generated which overexpress the polypeptide. Alternatively, the absence of the polypeptide in <<knock-out>> mice permits the study of the effects that loss of protein has on a cell in vivo. Knock-out mice also provide a model for the development of cancers.

Methods for producing knockout animals have been described previously. The production of conditional knockout animals, in which the gene is active until knocked out at the desired time is also known by those of ordinary skill in the art.

As noted above, transgenic animals and cell lines derived from such animals may find use in certain testing experiments. In this regard, transgenic animals and cell lines capable of expressing a tumor-specific or tumor-associated gene may be exposed to test substances. These test substances can be screened for the ability to reduce overexpression of the gene or impair the expression or function of a protein encoded by the gene.

In another embodiment, the invention provides a method for assaying expression of EST's utilizing microarrays comprising antibodies to the tumor-associated EST's of the invention.

In another embodiment, the invention provides a method for assaying for tumor EST's utilizing microarrays containing polypeptides or fragments thereof encoded and expressed by the tumor-associated EST's of the invention.

In another embodiment, the invention provides a method for assaying for tumor-associated EST's utilizing microarrays comprising nucleic acids specific for the tumor-related EST's of the invention.

The newly developed technique of nucleic acid analysis via microchip technology is also applicable to the present invention. In this technique, literally thousands of distinct oligonucleotide probes are built up in an array on a silicon chip. Nucleic acid to be analyzed is fluorescently labeled and hybridized to the probes on the chip. It is also possible to study nucleic acid-protein interactions using these nucleic acid microchips. Using this technique one can determine the presence of a sequence or expression levels of a gene of interest. The method is one of parallel processing of many, even thousands, of probes at once and can tremendously increase the rate of analysis.

It is also known in to persons of ordinary skill in the art that microchip technology is applicable to screening large numbers of samples by detecting antibody/antigen interactions. Utilizing cell type specific transcripts detected with the methods of the present invention, large numbers of cells from different stages of expression can be screened for expression of antigens. For a general description, see e.g., U.S. Pat. No. 6,379,895.

The nucleic acid, protein or antibody to the protein encoded by the nucleic acid may also be incorporated on a microarray. The preparation and use of microarrays are well known in the art. Generally, the microarray may contain the entire nucleic acid or protein, or it may contain one or more fragments of the nucleic acid or protein. Similarly, the microarray may contain an antibody or only the portion of the antibody necessary for binding antigen. It is contemplated by the invention that single chain antibodies may be utilized in the detection of tumor antigen or portions thereof. Suitable nucleic acid fragments may include at least 17 nucleotides, at least 21 nucleotides, at least 30 nucleotides or at least 50 nucleotides of the nucleic acid sequence, particularly where the nucleic acid marker comprises a coding sequence. Suitable protein fragments may include at least 4 amino acids, at least 8 amino acids, at least 12 amino acids, at least 15 amino acids, at least 17 amino acids or at least 20 amino acids.

In another embodiment, the invention provides methods for vaccinating an animal with tumor-associated polypeptides of the invention as an immunogen. A method of vaccination can comprise administering at least a fragment of a polypeptide encoded by the tumor-associated markers of the present invention. Methods for the administration of such fragments of a peptide are known to a person of ordinary skill in the art and can include administering additional peptide sequences as an adjuvant. In a preferred embodiment, the peptides are administered under conditions which will elicit a cytotoxic T-cell response to a tumor expressing a tumor-associated marker described in the present invention.

Cytotoxic T Lymphocytes (CTL) are an important means by which a mammalian organism defends itself against cancer. Functional studies of viral and tumor-associated T cells have confirmed that a minimal cytotoxic epitope consisting of a peptide of 8-12 amino acids can prime an antigen presenting cell to be lysed by CD8⁺ CTL, as long as the antigen presenting cell presents the epitope in the context of the correct MHC molecule. It is contemplated that the immunogen may comprise a minimal cytotoxic epitope on the tumor marker polypeptide. Minimal cytotoxic epitopes generally have been most effective when administered in the form of a lipidated peptide together with a helper CD4 epitope. Peptides administered alone, however, also can be highly effective.

As used herein, the singular form “a”, “an”, “said” and “the” include plural references unless the context clearly indicates otherwise. For example, a reference to a “cell” would include a plurality of cells.

As used herein, the terms “diagnosing” or “prognosing,” as used in the context of neoplasia, are used to indicate 1) the classification of lesions as neoplasia, 2) the determination of the severity of the neoplasia, or 3) the monitoring of the disease progression, prior to, during and after treatment.

“Encode”. A polynucleotide is said to “encode” a polypeptide if, in its native state or when manipulated by methods well known to those skilled in the art, it can be transcribed and/or translated to produce the mRNA for and/or the polypeptide or a fragment thereof. The anti-sense strand is the complement of such a nucleic acid, and the encoding sequence can be deduced therefrom.

“Isolated” or “substantially pure”. An “isolated” or “substantially pure” nucleic acid (e.g., an RNA, DNA or a mixed polymer) is one which is substantially separated from other cellular components which naturally accompany a native human sequence or protein, e.g., ribosomes, polymerases, many other human genome sequences and proteins. The term embraces a nucleic acid sequence or protein which has been removed from its naturally occurring environment, and includes recombinant or cloned DNA isolates and chemically synthesized analogs or analogs biologically synthesized by heterologous systems.

As used herein, the terms “tumor-associated marker” and “stress-associated marker” are meant to include nucleic acids or fragments thereof and polypeptides or fragments thereof that are specifically disclosed herein as associated with the indicated phenotype, as well as other nucleic acids or polypeptides or fragments thereof that comprise said polypeptides and nucleic acids and fragments thereof that can be detected with the methods of the present invention and are not known in the prior art to be associated with the particular phenotype.

As used herein, phenotype associated “marker expression” is meant to include the expression of all or a fragment of a specific (e.g., tumor-specific) or associated (e.g., tumor-associated) marker. Thus, as will be recognized by those of ordinary skill in the art, detection of marker expression is meant to include all known methods for detecting of gene expression, including but not limited to e.g. detecting the expression of an mRNA or fragment thereof (e.g., an EST) for the marker or detecting the expression of a polypeptide or fragment thereof encoded by a tumor associated marker of the invention. Polypeptide or fragments thereof can be detected by antibodies which specifically bind to the polypeptide or fragment thereof and allow its detection in various assay as known in the art such as Western blots, ELISA and the like.

The practice of the present invention employs, unless otherwise indicated, conventional techniques of chemistry, molecular biology, microbiology, recombinant DNA, genetics, immunology, cell biology, cell culture and transgenic biology, which are within the skill of the art.

General Methods

MTC panels. We used CLONTECH Multiple Tissue cDNA (MTC™) panels, which contain sets of normalized first-strand cDNA generated using CLONTECH Premium RNAT™ from different human tumors and normal tissues. These tissue-specific first strand cDNA's were used as templates in conjunction with tissue-specific tumor EST-derived primers in PCR studies to determine if tumor-associated EST's detected with the methods of the present invention were The following panels were used: Human Tumor MTC Panel (K1422-1), Human MTC Panel I (K1420-1), Human MTC Panel II (K1421-1), Human Immune System MTC Panel (K1426-1), and Human Fetal MTC Panel (K1425-1).

PCR analysis. PCR of genomic DNA was carried out in 25 μl of the following reaction mixture: 67 mM Tris-HCl (pH 8.9), 4 mM MgCl₂, 16 mM (NH₄)SO₄, 10 mM 2-mercaptoethanol, 0.1 mg/ml BSA, 200 μM (each) dNTP, specific forward and reverse primers (10 pmol each), 2.5U Taq polymerase, and 500 ng of genomic DNA. The samples were incubated in a PTC-200 thermocycler (MJ Research, USA) for the total of 35 cycles. Each cycle consisted of 30 s at 95° C., 30 s at 56° C. for forv/rev16 or at 58° C. for forw/rev8, forw/rev19, and forw/rev28, and 1 min at 72° C. DNA primers for PCR sequencing and the size of fragments generated for each cluster sequence were as follows:

Hs.154173:

forward16: (SEQ ID NO:1) 5′-TCT TTC TTG ATG AAT TAT CTT ATG-3′; reverse16: (SEQ ID NO:2) 5′-ACA CAC CCT CAT TCC CGC-3′; fragment size: 443 bp.

Hs.133294:

forward8: (SEQ ID NO:3) 5′-GTC AAC CTT CTC ATC TTC CTC-3′; reverse8: (SEQ ID NO:4) 5′-CAG GAA GTT GGG TAGATG TG-3′; fragment size: 1) 412 bp fragment size: 2) 1084 bp.

Hs.67624:

forward19:(SEQ ID NO:5) 5′-TAA TTG CAT TCT TCA AAA TTC TAC-3′;

reverse19: (SEQ ID NO:6) 5′-GCT TCG CAC CAT TGAATA AAC-3′; fragment size: 315 bp.

Hs.133107:

forward 28: (SEQ ID NO:7) 5′-TAC ATA GTT GTT ATC TTA AGG TG-3′; reverse 28: (SEQ ID NO: 8) 5′-TGG GAA TTC TAT ACT TTT GAC-3′; fragment size: 344 bp.

The expression of nucleotide sequences under study was analyzed in different tissues using CLONTECH cDNA panels and Titanium Taq PCR kit ( K1915-1). Reaction mixtures of a 25-μl volume were prepared according to the manufacturer's instructions for cDNA panels. PCR was carried out under the following conditions: 1 min at 95° C., 35 cycles consisting of 30 s at 95° C., 30 s at 56° C., for forw/rev16 or at 58° C., for forw/rev8, forw/rev19, or forw/rev28, and. 1 min at 68° C. The terminal stage of the reaction was 5 min at 68° C.

Electrophoresis. The amplification products were separated by electrophoresis in 2% agarose gel and detected by staining with ethidium bromide. 8 μl of PCR mixture was taken per lane.

Computer programs. Homology searches were performed using BLAST computer programs on a NCBI server (www.ncbi.nlm.nih.gov). Exon-intron boundaries and putative gene elements were predicted using program tools using techniques well known in the art and described in detail for example at the WebGene server (http://www.itba.mi.cnr.itlwebgene/) and on the search engine of Baylor College of Medicine. (http://kiwi.imgen.bcm.tmc.edu:8088/search-launcher/launcher.html).

Determination of exon-intron boundaries are indicative of genes as transcribed genomic units producing pre-mRNA spliced during RNA maturation.

The present invention is described by reference to the following Examples, which are offered by way of illustration and are not intended to limit the invention in any manner. Standard techniques well known by persons of ordinary skill in the art and/or the techniques specifically described herein were utilized.

### EXAMPLE 1

Utilizing publicly available EST sequence data and HSAnalyst, available clusters were organized into the ranges shown in Table 1. The software utilized in this example made possible the arrangement of sub ranges exponentially (e.g., sub ranges with exponents 1-2,3-4, 5-8,9-16) or linearly (sub ranges with factors 1-10, 11-20, 21-30). In this Example, the sub ranges were arranged linearly. Totally, 2681 libraries were classified as “tumor” libraries, while 1087 libraries were classified as “normal”. The supplemental database resulting from this differential comparison contained 921,237 “tumor” ESTs and 810,097 “normal” ESTs. Of these, 83 EST clusters were identified as putative tumor markers, possessing a percentage of tumor-specific EST's/total EST's of at least 90%. The classes of tumor related EST clusters revealed by the methods of the present invention were further classified into five distinct categories based on information provided about the sequences in the public databases, as detailed below in Tables 3-6. The clusters found to be tumor related included non-coding mRNAs, non-coding mRNAs with strict tumor specific expression, genes that encode proteins with weak homology to known proteins (as used herein, “weak refers to statistically significant homology that is not indicative of function or inclusion in the same gene family), genes that encode known proteins and genes that encode known proteins with a tumor associated expression. In some instances, EST clusters are tumor specific, not being expressed in the normal EST libraries. In other instances, the tumor EST's detected are tumor related, i.e., expressed at significantly higher levels in tumor cells versus normal cell sources. Table 1 represents an analysis of the number of tumor-associated EST's observed with the methods of the present invention.

Table I.

| | | | | Number of tumor-specific | | | |
|---|---|---|---|---|---|---|---|
| Sub-range of | EST number | | | clusters at threshold, %* | | | |
| # of EST's | # EST's per | # clusters | Tumor specific | >90% | | 100% | |
| per cluser | sub-range | per sub-range | EST's, % | Observed | Expected | Observed | Expected |
| 1-2 | 59111 | 44373 | 42% | 18342 | 23073 | 18342 | 23073 |
| 3-4 | 45400 | 13401 | 35% | 1880 | 1884 | 1880 | 1884 |
| 5-8 | 53569 | 8742 | 37% | 567 | 279 | 567 | 172 |
| 9-16 | 63421 | 5407 | 39% | 168 | 5 | 99 | 4 |
| 17-32 | 83968 | 3607 | 41% | 45 | 0 | 17 | 0 |
| 33-64 | 176845 | 3762 | 43% | 16 | 0 | 2 | 0 |
| 65-128 | 349008 | 3790 | 45% | 10 | 0 | 2 | 0 |
| 129-256 | 460493 | 2588 | 47% | 8 | 0 | 0 | 0 |
| 257-512 | 339482 | 975 | 50% | 3 | 0 | 0 | 0 |
| 513-1024 | 208171 | 303 | 53% | 1 | 0 | 0 | 0 |
| 1025-2048 | 130524 | 96 | 57% | 0 | 0 | 0 | 0 |
| 2049-4096 | 95180 | 36 | 60% | 0 | 0 | 0 | 0 |
| 4097-8192 | 49804 | 10 | 66% | 0 | 0 | 0 | 0 |
| 8193-16384 | 14725 | 1 | 67% | 0 | 0 | 0 | 0 |

An exemplary method for detecting tumor-associated EST's comprised retrieving sequence data on EST's from all available EST's, arranging the EST's into individual clusters based on homology, identifying EST's expressed in tumor cells and, for each cluster, calculating the percentage of the number of ESTs expressed in tumor cells to all EST's contained in the cluster. A threshold value for the percentage of the number of ESTs expressed in tumor cells to all ESTs for each cluster was chosen to identify tumor related clusters. In one example, the percentage of tumor-derived EST's to normal EST's per cluster was a user-defined threshold of at least 90%. Clusters having a percentage of EST's expressed in tumor cells to all EST's for a cluster greater than the threshold value were considered as tumor-associated. Thus, tumor-associated markers represent those nucleic acid or polypeptide or fragments thereof that comprise at least 90% of the sequences in an EST cluster. Some sequences observed were markers that represented nucleic acid or polypeptides or fragments thereof that comprised 100% of the sequences in a cluster.

In Table I, there are shown the results of detection of clusters observed at different ranges, with the number of observed tumor related clusters observed versus the number calculated or expected. Clusters were sorted into ranges on a linear basis in this example.

Using global analysis of cluster data with the methods of the present invention, it has been demonstrated that the sequences of Table 2 represent tumor-associated sequences.

TABLE II

| | | | SURFACE, IF KNOWN | REFERENCE | REFERENCE |
|---|---|---|---|---|---|
| | | | KNOWN TUMOR | NUCLEOTIDE | PROTEIN |
| UNIGENE ID | GENE NAME | TUMOR TYPES | MARKER INDICATED | SEQUENCE | SEQUENCE(S) |
| Hs.203 | CCKBR (Cholecystoki-nin B receptor) | Choriocarcinoma, glioma, germ cell | SURFACE | SEQ. ID NO: 9 | SEQ. ID NO: 10 |
| | | tumors, lung carcinoma, | | | |
| | | teratocarcinoma | | | |
| Hs.419 | DLX2 (Distal-less homeo box 2 | small cell lung carcinoma, pancreatic | | SEQ. ID NO: 11 | SEQ. ID NO: 12 |
| | | carcinoma, intestinal | | | |
| | | carcinoma, ovary carcinoma | | | |
| Hs.560 | APOBEC1 ApolipoproteinB mRNA | colon carcinoma; B-cell chronic | | SEQ. ID NO: 13 | SEQ. ID NO: 14 |
| | editing enzyme, cata-lytic | lymphotic leukemia; | | | |
| | polypeptide 1 | | | | |
| Hs.575 | ALDH3A1 | Pancreatic carcinoma, glioma, | | SEQ. ID NO: 15 | SEQ. ID NO: 16 |
| | (Aldehydedehydrogenase 3 | cervical carcinoma , lung | | | |
| | family, member A1) | carcinoma, uterine carcinoma, | | | |
| | | germ cell tumors, gastric carcinoma, | | | |
| | | colon carcinoma, salivary gland | | | |
| | | carcinoma, bladder carcinoma | | | |
| Hs.1085 | GUCY2C Guanylate cyclase 2C | stomach carcinoma, colon carcinoma | SURFACE, | SEQ. ID NO: 17 | SEQ. ID NO: 18 |
| | (heat stable enterotoxin | | KNOWN TUMOR | | |
| | receptor) | | MARKER | | |
| Hs.1149 | LMO1 LIM domain only 1 | Glioma, retinoblastoma, lung | KNOWN MARKER | SEQ. ID NO: 19 | SEQ. ID NO: 20 |
| | (rhombotin 1) | carcinoid tumors, pancreatic | FOR LEUKEMIA | | |
| | | insulinoma | | | |
| Hs.1619 | ASCL1 Achaete-scute complex- | neuroblastoma, glioma lung carcinoid tumors, | KNOWN TUMOR | SEQ. ID NO: 21 | SEQ. ID NO: 22 |
| | like 1 (Drosophila-like) | germ cell tumors, kidney tumor, | MARKER | | |
| | | medulloblastoma, ovary tumors | | | |
| Hs.1854 | KCNA4 Potassium voltage-gated | lung carcinoid tumors, lung carcinomas | SURFACE | SEQ. ID NO: 23 | SEQ. ID NO: 24 |
| | channel, shaker-related | | | | |
| | subfamily, member 4 | | | | |
| Hs.1925 | DSG3 Desmoglein 3 (pemphigus | lung carcinomas, pancreatic carcinoma | RARANEOPLASTIC | SEQ. ID NO: 25 | SEQ. ID NO: 26 |
| | vulgaris antigen) | | MARKER | | |
| Hs.2266 | CHRNA1 Cholinergic receptor, | Rhabdomyosarcoma | SURFACE | SEQ. ID NO: 27 | SEQ. ID NO: 28 |
| | nicotinic, alpha polypeptide | | | | |
| | 1 (muscle) | | | | |
| Hs.2693 | GLI Glioma-associated | Rhabdomyosarcoma, germ cell tumors, | KNOWN MARKER | SEQ. ID NO: 29 | SEQ. ID NO: 30 |
| | oncogene homolog (zinc finger | leiomyosarcoma, ovarian tumors, melanoma, | FOR GLIOMA | | |
| | protein) | burkitt lymphoma | | | |
| Hs.2860 | POU5F1 POU domain, class 5, | gastric carcinoma, germ cell tumors, uterus | KNOWN MARKER | SEQ. ID NO: 31 | SEQ. ID NO: 32 |
| | transcription factor 1 | carcinoma, ovarian tumors, teratocarcinoma | FOR GERM CELL | | |
| Hs.2928 | SLC7A1 Solute carrier family | melanoma, glioma, rhabdomyosarcoma | SURFACE | SEQ. ID NO: 33 | SEQ. ID NO: 34 |
| | 7 (cationic amino acid | neuroblastoma, colon carcinomas, lymphoma | | | |
| | transporter, y + system), | | | | |
| | member 1 | | | | |
| Hs.3057 | ZNF74 Zinc finger protein 74 | cervical carcinoma , leiomyosarcoma, | | SEQ. ID NO: 35 | SEQ. ID NO: 36 |
| | (Cos52) | rhabdomyosarcoma glioma , teratocarcinoma | | | |
| | | neuroblastoma , prostate carcinoma, colon | | | |
| | | carcinoma , choriocarcinoma, bladder | | | |
| | | transitional cell papilloma | | | |
| Hs.3104 | KIAA0042 (KIAA0042 gene | Leiomyosarcoma, testicular cancer, prostate | | SEQ. ID NO: 37 | SEQ. ID NO: 38 |
| | product) | carcinoma, bladder carcinoma, kidney | | | |
| | POM1 | hypernephroma, ovarian tumors, lung | | | |
| | | carcinoma | | | |
| Hs.5366 | EPS8R3 Epidermal growth | Colon carcinoma, kidney tumors, germ cell | | SEQ. ID NO: 39 | SEQ. ID NO: 40 |
| | factor receptor pathway | tumors, stomach carcinoma | | | |
| | substrate 8 related protein 3 | | | | |
| Hs.6168 | KIAA0703 (KIAA0703 gene | Pancreatic carcinoma, colon carcinoma, | | SEQ. ID NO: 41 | SEQ. ID NO: 42 |
| | product) | bladder transitional cell papilloma, ovarian | | | |
| | POM2 | carcinoma, breast carcinoma , lung carcinoma | | | |
| Hs.30743 | PRAME Preferentiallyexpressed | Brain neuroblastoma, melanoma, lung | KNOWN TUMOR | SEQ. ID NO: 43 | SEQ. ID NO: 44 |
| | antigen in melanoma | carcinoma , small intestine carcinoma, | MARKER FOR | | |
| | | retinoblastoma, leiomyosarcoma, uterus | MELANOMA | | |
| | | carcinoma, choriocarcinoma ,kidney | | | |
| | | carcinoma, ovarian carcinoma, bresat | | | |
| | | carcinoma, germ cell tumor, esophageal | | | |
| | | squamous cell carcinoma, colon juvenile | | | |
| | | granulosa tumor, cervical carcinoma | | | |
| Hs.30751 | LOC55924 Hypothetical protein | Retinoblastoma, rhabdomyosarcoma, prostate | | SEQ. ID NO: 45 | SEQ. ID NO: 46 |
| | LOC55924 POM3 | carcinoma, Burkitt lymphoma | | | |
| Hs.36793 | SLC12A8 Solute carrier family | Lymphoma, colon, ovarian, stomach, prostate | SURFACE | SEQ. ID NO: 47 | SEQ. ID NO: 48 |
| | 12 (potassium/chloride | endometrial and hepatic carcinomas | | | |
| | transporters), member 8 | | | | |
| Hs.37045 | PTH Parathyroid hormone | parathyroid tumor | KNOWN TUMOR | SEQ. ID NO: 49 | SEQ. ID NO: 50 |
| | | | MARKER | | |
| Hs.37107 | MAGEA4 Melanoma antigen, | intestine duodenal carcinoma, glioma, | KNOWN TUMOR | SEQ. ID NO: 51 | SEQ. ID NO: 52 |
| | family A, 4 | pharynx squamous cell, uterus, ovarian, | MARKER FOR | | |
| | | melanoma | MELANOMA | | |
| Hs.37110 | MAGEA9 Melanoma | Lung carcinoma, bladder transitional cell | KNOWN TUMOR | SEQ. ID NO: 53 | SEQ. ID NO: 54 |
| | antigen, familyA, 9 | papilloma, T cell leukemia, genitourinary | MARKER FOR | | |
| | | tract transitional cell tumors | MELANOMA | | |
| Hs.46452 | SCGB2A2 Secretoglobin, family | lung carcinoma | SURFACE | SEQ. ID NO: 55 | SEQ. ID NO: 56 |
| | 2A, member 2 | | | | |
| Hs.48956 | GJB6 Gap junction protein, | glioma, prostate carcinoma, uterus | SURFACE | SEQ. ID NO: 57 | SEQ. ID NO: 58 |
| | beta 6 (connexin 30) | carcinoma, pancreatic carcinoma, skin | | | |
| | | squamous cell carcinoma | | | |
| Hs.49605 | ESTs, Weakly similar to | melanoma | | SEQ. ID NO: 59 | SEQ. ID NO: 60 |
| | hypothetical protein FLJ22184 | | | | |
| | [Homo sapiens] | | | | |
| | POM4 | | | | |
| Hs.53563 | COL9A3 Collagen, type IX, | melanoma, choriocarcinoma, B-cell chronic | | SEQ. ID NO: 61 | SEQ. ID NO: 62 |
| | alpha 3 | lymphotic leukemia, germ cell, uterus serous | | | |
| | | carcinoma, stomach carcinoma, retinoblastoma | | | |
| | | sarcoma, glioma, cervical carcinoma | | | |
| Hs.54424 | HNF4A Hepatocyte nuclear | Kidney tumors, germ cell tumors, colon | | SEQ. ID NO: 63 | SEQ. ID NO: 64 |
| | factor 4, alpha | carcinoma | | | |
| Hs.54567 | PAX1 Paired box gene 1 | leiomyosarcoma | | SEQ. ID NO: 65 | SEQ. ID NO: 66 |
| Hs.66357 | POM5 | Endometrial, pancreatic, lymphoma, lung B- | | SEQ. ID NO: 67 | SEQ. ID NO: 68 |
| | | cell chronic lymphocytic leukemia | | | |
| Hs.67397 | HOXA1 Homeobox A1 | melanoma, teratocarcimoma, germ cell tumors | | SEQ. ID NO: 69 | SEQ. ID NO: 70 |
| | | stomach carcinoma, hypernephroma, bladder | | | SEQ. ID NO: 71 |
| | | carcinoma | | | SEQ. ID NO: 72 |
| Hs.67624 | POM6 | germ cell tumors | | SEQ. ID NO: 73 | SEQ. ID NO: 74 |
| Hs.68864 | Membrane-bound phosphatidic | B-cell chronic lymphocytic leukemia, colon, | SURFACE | SEQ. ID NO: 75 | SEQ. ID NO: 76 |
| | acid-selective phospholipasE | stomach, pancreatic carcinomas | | | |
| | A1 | | | | |
| Hs.73893 | DRD2 Dopamine receptor D2 | Lung carcinoma, neuroblastoma, glioma, | SURFACE | SEQ. ID NO: 77 | SEQ. ID NO: 78 |
| | | pancreas carcinoma, rhabdomyosarcoma | | | |
| Hs.73952 | PRH2 Proline-rich protein | Nervous tumors, colon carcinoma, | SECRETED | SEQ. ID NO: 79 | SEQ. ID NO: 80 |
| | HaeIII subfamily 2 | head and neck squamous cell carcinoma | | | |
| Hs.74126 | FABP6Fatty acid binding | Lymphoma, uterus carcinoma, kidney | | SEQ. ID NO: 81 | SEQ. ID NO: 82 |
| | protein 6, ilealgastrotropin) | Carcinoma, lung carcinoid tumors, ovarian | | | |
| Hs.79414 | PDEF Prostate epithelium- | Pancreatic, colon, endometrial, breast, | KNOWN MARKER- | SEQ. ID NO: 83 | SEQ. ID NO: 84 |
| | specific Ets transcription | lung, ovarian, stomach, prostate carcinomas | BREAST CARCINOMA | | |
| | factor | and glioma | POSSIBLYPROSTATIC | | |
| | | | CARCINOMA) | | |
| Hs.86232 | GDF3 Growth differentia-tion | germ cell tumors, neuroepithelial tumors | Embryonal | SEQ. ID NO: 85 | SEQ. ID NO: 86 |
| | factor 3 | | carcinoma stem | | |
| | | | cell-associated | | |
| | | | marker; Possibly | | |
| | | | GERM CELL TUMORS | | |
| Hs.87225 | CTAG2 Cancer/testis antigen 2 | choriocarcinoma, breast carcinoma, | KNOWN TUMOR | SEQ. ID NO: 87 | SEQ. ID NO: 88 |
| | | endometrium carcinoma, melanoma, stomach | MARKER | | |
| | | carcinoma | | | |
| Hs.89143 | POM7 | ovarian tumors | | SEQ. ID NO: 89 | SEQ. ID NO: 90 |
| Hs.89605 | CHRNA3 Cholinergic receptor, | neuroblastoma, lung carcinoma, small | SURFACE | SEQ. ID NO: 91 | SEQ. ID NO: 92 |
| | nicotinic, alpha polypeptide3 | intestine carcinoma | | | |
| Hs.97258 | POM8 similar to S29539 | Pancreas, endometrial, ovarian carcinomas | | SEQ. ID NO: 93 | SEQ. ID NO: 94 |
| | ribosomal protein L13a, | lung carcinoid tumors and germ cell tumors | | | |
| | cytosolic | | | | |
| Hs.97283 | POM9 | ovarian tumors | | SEQ. ID NO: 95 | SEQ. ID NO: 96 |
| Hs.97860 | KIAA1484 KIAA1484 protein | Ovarian carcinoma, retinoblastoma, | | SEQ. ID NO: 97 | SEQ. ID NO: 98 |
| | | endometrium carcinoma | | | |
| Hs.98988 | POM10 Homo sapiens, clone | germ cell tumors, hypernephroma, ovarian | | SEQ. ID NO: 99 | SEQ. ID NO: 100 |
| | IMAGE:4425111, mRNA, partial | tumors, colon, uterus, stomach, pancreas | | | |
| | cds | skin squamous cell carcinomas | | | |
| Hs.99624 | POM11 | parathyroid tumor, | | SEQ. ID NO: 101 | SEQ. ID NO: 102 |
| | | ovarian tumor, Stomach carcinoma | | | |
| Hs.99960 | MS4A3 Membrane-spanning 4- | Lung carcinoma, chronic myelogenous | SURFACE | SEQ. ID NO: 103 | SEQ. ID NO: 104 |
| | domains, subfamily A, member | leukemia, prostate carcinoma | | | |
| | 3 (hematopoieticcell- | | | | |
| | specific) | | | | |
| Hs.103504 | ESR2 Estrogen receptor 2 (ER | germ cell tumors, lung carcinoma, | KNOWN TUMOR | SEQ. ID NO: 105 | SEQ. ID NO: 106 |
| | beta) | neuroblastoma | MARKER | | |
| Hs.103707 | MUC5AC Mucin 5, subtypes A | COLON, PANCREATIC, STOMACH CARCINOMAS, | SURFACE, | SEQ. ID NO: 107 | SEQ. ID NO: 108 |
| | and C, tracheobron- | LUNG TUMORS | MARKER FOR | | |
| | chial/gastric | | COLON AND | | |
| | | | GASTRIC | | |
| | | | CARCINOMAS | | |
| Hs.104073 | POM12 | Colon, stomach carcinoma | | SEQ. ID NO: 109 | SEQ. ID NO: 110 |
| Hs.104115 | ZNF10 Zinc finger protein 10 | parathyroid, lung carcinoid, nervous cell | | SEQ. ID NO: 111 | SEQ. ID NO: 112 |
| | (KOX1) | tumors, adrenal cortex carcinoma, germ cell | | | |
| | | tumors, uterus tumor, multiple myeloma | | | |
| Hs.105484 | REG-IV Regenerating gene type | Prostate, duodenal, colon and stomach | | SEQ. ID NO: 113 | SEQ. ID NO: 114 |
| | IV | carcinomas, B-cell chronic lymphocytic | | | |
| | | leukemia, acute myelogenous leukemia | | | |
| Hs.105667 | POM13 | ovarian tumors | | SEQ. ID NO: 115 | SEQ. ID NO: 116 |
| Hs.105924 | DEFB4 Defensin, beta 4 | Head and neck carcinoma | SECRETED | SEQ. ID NO: 117 | SEQ. ID NO: 118 |
| Hs.112341 | PI3 Protease inhibitor 3, | Glioma, B-cell chronic lymphocytic leukemia, | | SEQ. ID NO: 119 | SEQ. ID NO: 120 |
| | skin-derived (SKALP) | uterus, lung and colon carcinomas, ovarian, | | | |
| | | prostate, colon carcinomas, bladder, nervous | | | |
| | | cell and placenta tumors | | | |
| Hs.113262 | HTR45 hydroxytryptamine | Schwannona | SURFACE | SEQ. ID NO: 121 | SEQ. ID NO: 122 |
| | (serotonin) receptor 4 | | | SEQ. ID NO: 123 | SEQ. ID NO: 124 |
| Hs.114905 | ERN2 (ER to nucleus | Stomach, colon, pancreatic carcinoma | | SEQ. ID NO: 125 | SEQ. ID NO: 126 |
| | signalling 2) | | | | |
| Hs.117938 | COL17A1 Collagen, type XVII, | glioma, pancreas, lung, colon, | | SEQ. ID NO: 127 | SEQ. ID NO: 128 |
| | alpha 1 | nasopharyngeal, stomach carcinomas, germ | | | |
| | | cell, bladder, uterus tumors, | | | |
| | | leiomyosarcoma | | | |
| Hs.122310 | POM14 | parathyroid tumor | | SEQ. ID NO: 129 | SEQ. ID NO: 130 |
| Hs.123094 | SALL1 Sal-like 1 (Drosophila) | Retinoblastoma, germ cell tumors, glioma | | SEQ. ID NO: 131 | SEQ. ID NO: 132 |
| Hs.123993 | POM15 | Glioma, colon carcinoma, lung carcinoid | | SEQ. ID NO: 133 | SEQ. ID NO: 134 |
| | Weakly similar to T00366 | tumors, parathyroid tumor | | | |
| | hypothetical protein KIAA0669 | | | | |
| Hs.124173 | POM16 | parathyroid tumor | | SEQ. ID NO: 135 | SEQ. ID NO: 136 |
| Hs.124568 | POM17 | COLON CARCINOMA | | SEQ. ID NO: 137 | |
| Hs.125293 | POM18 | Glioma, lung | | SEQ. ID NO: 138 | SEQ. ID NO: 139 |
| | | carcinoma, kidney tumors, germ cell tumors | | | |
| | | parathyroid tumor, stomach carcinoma, ovary | | | |
| | | carcinoma | | | |
| Hs.126566 | POM19 | Colon carcinoma | | SEQ. ID NO: 140 | SEQ. ID NO: 141 |
| Hs.126869 | POM20 | LUNG CARCINOID TUMORS, germ cell tumor | | SEQ. ID NO: 142 | SEQ. ID NO: 143 |
| Hs.127144 | POM21 | Colon carcinoma | | SEQ. ID NO: 144 | SEQ. ID NO: 145 |
| Hs.127383 | POM22 | Colon carcinoma | | SEQ. ID NO: 146 | SEQ. ID NO: 147 |
| Hs.127476 | POM23 | Lung carcinoid tumors, glioma, kidney | | SEQ. ID NO: 148 | SEQ. ID NO: 149 |
| | Highly similar to BTG2_HUMAN | tumors, chondrosarcoma, germ cell tumors, | | | |
| | BTG2 PROTEIN PRECURSOR | Ewing's sarcoma | | | |
| Hs.128001 | POM24 | COLON CARCINOMA | | SEQ. ID NO: 150 | SEQ. ID NO: 151 |
| | | | | | SEQ. ID NO: 152 |
| Hs.128115 | POM25 Homo sapiens cDNA | germ cell,lung carcinoid and kidney tumors, | | SEQ. ID NO: 153 | SEQ. ID NO: 154 |
| | FLJ32217 fis, clone | glioma, melanoma | | | |
| | PLACE6003771 | | | | |
| Hs.128326 | POM26 | germ cell tumors | | SEQ. ID NO: 155 | SEQ. ID NO: 156 |
| Hs.128398 | POM27 | Lung carcinoid tumors | | SEQ. ID NO: 157 | |
| Hs.128436 | POM28, Moderately similar to | Lung carcinoid tumors | | SEQ. ID NO: 158 | SEQ. ID NO: 159 |
| | putative secreted protein | | | | |
| | [Homo sapiens] | | | | |
| Hs.128437 | POM29, Weakly similar to | Lung carcinoid tumors, kidney tumors, | | SEQ. ID NO: 160 | SEQ. ID NO: 161 |
| | S33477 hypothetical protein 1- | cervical carcinoma | | | |
| | rat | | | | |
| Hs.128907 | POM30, Weakly similar to | LUNG CARCINOID TUMORS | | SEQ. ID NO: 162 | SEQ. ID NO: 163 |
| | orthopedia homolog | | | | |
| | (Drosophila); orthopedia | | | | |
| | (Drosphila) homolog; | | | | |
| | orthopedia (Drosophila) | | | | |
| | homolog; Orthopedia, homolog | | | | |
| | of Drosophila gene [Homo | | | | |
| | sapiens] [H.sapiens | | | | |
| Hs.129040 | POM31 | parathyroid tumor, lung carcinoid tumors | | SEQ. ID NO: 164 | SEQ. ID NO: 165 |
| Hs.129108 | POM32 | Lung carcinoid tumors | | SEQ. ID NO: 166 | SEQ. ID NO: 167 |
| | clone IMAGE:2337282 | | | | |
| Hs.129302 | POM33 | lung carcinoma, germ cell tumors | | SEQ. ID NO: 168 | SEQ. ID NO: 169 |
| Hs.129782 | MUC3B Mucin 3B | Pancreatic carcinoma, kidney tumors, colon | PROBABLY KNOWN | SEQ. ID NO: 170 | SEQ. ID NO: 171 |
| | | carcinoma choriocarcinoma, breast carcinoma | TUMOR MARKER | | |
| | | stomach tumor, head and neck tumor, lung | | | |
| | | tumor, ovary tumor | | | |
| Hs.131358 | POM34 | germ cell tumors, choriocarcinoma | | SEQ. ID NO: 172 | SEQ. ID NO: 173 |
| Hs.132370 | NOX1 NADPH oxidase 1 | colon carcinomas, glioma, lung carcinoid | | SEQ. ID NO: 174 | SEQ. ID NO: 175 |
| | | tumors, kidney tumors, breast carcinoma | | SEQ. ID NO: 176 | SEQ. ID NO: 177 |
| Hs.132576 | Paired box gene 9 | Lung carcinoma, parathyroid tumor, stomach | | SEQ. ID NO: 178 | SEQ. ID NO: 179 |
| | | carcinoma , head and neck carcinoma | | | |
| Hs.133081 | POM35 | Esophagus carcinoma, germ cell tumors, | | SEQ. ID NO: 180 | SEQ. ID NO: 181 |
| | Homo sapiens cDNA | glioma, lung carcinoma, chondrosarcoma, | | | |
| | FLJ25124 fis | uterus carcinoma | | | |
| Hs.133089 | DFFB DNA fragmentation | Lung carcinoid tumors, breast carcinoma, | | SEQ. ID NO: 182 | SEQ. ID NO: 183 |
| | factor, 40 kD, beta | colon carcinoma, nervous cell tumor, | | | |
| | polypeptide (caspase- | leiomioma, acute myelogenous leukemia, | | | |
| | activated DNase) | osteosarcoma | | | |
| Hs.133107 | POM36 | Ovary carcinoma, lung carcinoma, glioma | | SEQ. ID NO: 184 | SEQ. ID NO: 185 |
| Hs.133294 | POM37 | Uterus carcinoma, lung carcinoma, Ovary | | SEQ. ID NO: 186 | SEQ. ID NO: 187 |
| | | carcinoma, chronic myelogenous leukemia, | | | SEQ. ID NO: 188 |
| | | breast carcinoma, glioma, colon juvenile | | | |
| | | granulosa tumor, adrenal adenoma, prostate | | | |
| | | tumor, head and neck carcinoma | | | |
| Hs.133296 | POM38 | Ovary carcinoma, lung carcinoma | | SEQ. ID NO: 189 | SEQ. ID NO: 190 |
| Hs.133300 | POM39 | Breast carcinoma, ovary carcinoma, lung | | SEQ. ID NO: 191 | SEQ. ID NO: 192 |
| | | carcinoma | | | |
| Hs.133451 | POM40 | germ cell tumors, colon carcinoma | | SEQ. ID NO: 193 | SEQ. ID NO: 194 |
| Hs.135365 | POM41 | Pancreatic carcinoma, ovarian carcinoma, | | SEQ. ID NO: 195 | SEQ. ID NO: 196 |
| | | lung carcinoma | | | |
| Hs.140457 | POM42 | Kidney tumors, lung carcinoid tumorss, | | SEQ. ID NO: 197 | SEQ. ID NO: 198 |
| | | insulinoma, glioma, cervical carcinoma, | | | |
| | | stomach tumors | | | |
| Hs.142907 | POM43 Human BRCA2 region, | Lung carcinoid tumors, fibrotheoma, ovary | | SEQ. ID NO: 199 | SEQ. ID NO: 200 |
| | mRNA sequence CG011 | tumors, uterus tumors | | | |
| Hs.143507 | T T, brachyury homolog | Lung carcinoma, B-cell chronic lymphocytic | | SEQ. ID NO: 201 | SEQ. ID NO: 202 |
| | | leukemia, breast carcinoma, germ cell tumors | | | |
| Hs.143949 | POM44 | Colon carcinoma | | SEQ. ID NO: 203 | SEQ. ID NO: 204 |
| Hs.144063 | POM45 | Lung carcinoid tumorss | | SEQ. ID NO: 205 | SEQ. ID NO: 206 |
| Hs.144121 | POM46, Moderately similar to | glioma, lung carcinoma | | SEQ. ID NO: 207 | SEQ. ID NO: 208 |
| | hypothetical protein, MNCb- | | | | |
| | 123; hypothetical protein, | | | | |
| | MNCb-1231 | | | | |
| Hs.145327 | POM47 | chronic myalogenous leukemia, Ovary | | SEQ. ID NO: 209 | SEQ. ID NO: 210 |
| | | carcinoma, colon carcinoma, lung carcinoma | | | |
| | | head and neck carcinoma | | | |
| Hs.145340 | POM48 | lung carcinoma, Ovary carcinoma, head and | | SEQ. ID NO: 211 | SEQ. ID NO: 212 |
| | | neck carcinoma | | | |
| Hs.145356 | POM49 | Ovary carcinoma, lung carcinoma | | SEQ. ID NO: 213 | SEQ. ID NO: 214 |
| Hs.145357 | POM50 | Ovary carcinoma, breast carcinoma, head and | | SEQ. ID NO: 215 | SEQ. ID NO: 216 |
| | | neck carcinoma, lung carcinoma | | | |
| Hs.145489 | POM51 | Ovary carcinoma | | SEQ. ID NO: 217 | SEQ. ID NO: 218 |
| Hs.145492 | POM52 | Ovary carcinoma, lung carcinoma | | SEQ. ID NO: 219 | SEQ. ID NO: 220 |
| Hs.145493 | POM53 | Ovary carcinoma, uterus tumor | | SEQ. ID NO: 221 | SEQ. ID NO: 222 |
| Hs.145500 | POM54 | Ovary carcinoma, lung carcinoma | | SEQ. ID NO: 223 | SEQ. ID NO: 224 |
| Hs.145509 | POM55 | Lung carcinoma, ovary carcinoma, breast | | SEQ. ID NO: 225 | SEQ. ID NO: 226 |
| | | carcinoma, glioma, stomach carcinoma | | | |
| Hs.145661 | POM56 | Colon carcinoma | | SEQ. ID NO: 227 | SEQ. ID NO: 228 |
| Hs.145809 | POM57, Weakly similar to | Uterus carcinoma, stomach carcinoma, | | SEQ. ID NO: 229 | |
| | T31613 hypothetical protein | pancreatic carcinoma, placenta tumor | | | |
| | Y50E8A.i - Caenorhabditis | | | | |
| | elegans | | | | |
| Hs.146200 | POM58 | Ovary carcinoma, breast carcinoma, head and | | SEQ. ID NO: 230 | SEQ. ID NO: 231 |
| | | neck carcinoma | | | |
| Hs.147291 | POM59 | germ call tumors | | SEQ. ID NO: 232 | SEQ. ID NO: 233 |
| Hs.148661 | POM60 | Lung carcinoid tumors, germ cell tumors | | SEQ. ID NO: 234 | SEQ. ID NO: 235 |
| Hs.152290 | POM61, Highly similar to | Rhabdomyosarcoma, glioma, colon carcinoma | | SEQ. ID NO: 236 | SEQ. ID NO: 237 |
| | VIPS_HUMAN VASOACTIVE | | | | |
| | INTESTINAL POLYPEPTIDE | | | | |
| | RECEPTOR 2 PRECURSOR | | | | |
| | [H. sapiens] | | | | |
| Hs.152531 | HAND1 Heart and neural crest | Neuroblastoma, Schwannoma, germ cell tumors | | SEQ. ID NO: 238 | SEQ. ID NO: 239 |
| | derivatives expressed 1 | sarcoma | | | |
| Hs.153444 | POM62 | Lung carcinoid tumors, breast carcinoma | | SEQ. ID NO: 240 | SEQ. ID NO: 241 |
| Hs.352562 | POM63, | Teratocarcinoma, laposarcoma, | | SEQ. ID NO: 242 | SEQ. ID NO: 243 |
| | Homo sapiens cDNA FLJ33010 | pheochromocytoma, lung carcinoma, cervical | | | |
| | is, clone THYMU1000336 | carcinoma, chondrosarcoma, breast carcinoma, | | | |
| | UniGene cluster identifier | leiomioma, lymphoma, uterus tumor, head and | | | |
| | Hs.154173 has been retired | neck carcinomar, colon carcinoma, breast | | | |
| | current cluster Hs.352562 | carcinoma, melanoma, skin carcinoma, | | | |
| | | prostate tumor | | | |
| Hs.155981 | MSLN Mesothelin | Pancreas, prostate, cervical, liver, uterus, | KNOWN TUMOR | SEQ. ID NO: 244 | SEQ. ID NO: 245 |
| | | colon, stomach, head and neck and lung | MARKER FOR | | |
| | | carcinomas, choriocarcinoma, glioma, | CARCINOMAS | | |
| | | ovarian and uterus tumors, chondrosarcoma | | | |
| Hs.156213 | POM64 | Lung carcinoid tumors, head and neck | | SEQ. ID NO: 246 | SEQ. ID NO: 247 |
| | | carcinoma, colon carcinoma | | | |
| Hs.156499 | POM65 | Uterus tumors, Lymhomas and leukemias | | SEQ. ID NO: 248 | SEQ. ID NO: 249 |
| Hs.156637 | CBLC Cas-Br-M (murine) | stomach, lung, breast, colon, lung pancreas | | SEQ. ID NO: 250 | SEQ. ID NO: 251 |
| | ectropic retroviral | and head and neck carcinomas, glioma, | | | |
| | transforming sequence c | choriocarcinoma Uterus and carcinoid tumors | | | |
| Hs.156762 | POM66 | germ cell tumors | | SEQ. ID NO: 252 | SEQ. ID NO: 253 |
| Hs.156810 | POM67 Weakly similar to | Uterus carcinoma | | SEQ. ID NO: 254 | SEQ. ID NO: 255 |
| | EF11_HUMAN ELONGATION FACTOR | | | | |
| | 1-ALPHA 1 [H.sapiens] | | | | |
| Hs.156813 | POM6B (MGC10600) predicted | Melanoma, choriocarcinoma, germ cell tumor | | SEQ. ID NO: 256 | SEQ. ID NO: 257 |
| | protein MGC10600 | | | | |
| Hs.156843 | POM69 | Lung carcinoid tumors, germ cell tumors, | | SEQ. ID NO: 258 | SEQ. ID NO: 259 |
| | | melanoma | | | |
| Hs.156905 | KIAA1676 | germ cell and lung carcinoid tumors, Ewing's | | SEQ. ID NO: 260 | SEQ. ID NO: 261 |
| | | sarcoma, ovary, adrenal cortex and uterus | | | |
| | | carcinomas, retinoblastoma | | | |
| Hs.157205 | BCAT1 Branched chain | germ cell tumors, lung carcinoma, glioma, | | SEQ. ID NO: 262 | SEQ. ID NO: 263 |
| | aminotransfe-rase 1, | lymphoma, teratocarcinoma, rhabdomyosarcoma, | | | |
| | cytosolic | lung carcinoma, embryonal carcinoma, uterus | | | |
| | | tumor | | | |
| Hs.79707 | TNFRSF19L Tumor necrosis | Colon carcinoma, glioma, B-cell chronic | | SEQ. ID NO: 264 | SEQ. ID NO: 265 |
| | factor receptor superfamily, | lymphocytic leukemia, ovary tumors, germ | | | |
| | member 19-like | cell tumors, chondrosarcoma, neuroblastoma, | | | |
| | UniGene cluster indentifier | melanoma, stomach carcinoma, | | | |
| | Hs.158218 has been retired | leiomyosarcoma, renal cell carcinoma, uterus | | | |
| | now Hs.79707 | carcinoma, lung carcinoma, lymphoma, pre-B | | | |
| | | cell acute lymphoblastic leukemia | | | |
| Hs.158333 | PRSS7 Protease, serine, 7 | Glioma, breast carcinoma | | SEQ. ID NO: 266 | SEQ. ID NO: 267 |
| | (enterokinase) | | | | |
| Hs.158460 | CDK5R2 Cyclin-dependent | germ cell tumors, lung carcinoid tumors, | | SEQ. ID NO: 268 | SEQ. ID NO: 269 |
| | kinase 5, regulatory subunit | glioma, adrenal cortex carcinoma, lung | | | |
| | 2 (p39) | carcinoma, neuroblastoma | | | |
| Hs.158521 | POM70 | Kidney tumors, breast carcinoma | | SEQ. ID NO: 270 | SEQ. ID NO: 271 |
| Hs.160724 | POM71 | glioma, lung carcinoid tumors | | SEQ. ID NO: 272 | SEQ. ID NO: 273 |
| Hs.162717 | P0M72, | Choriocarcinoma, neuroblastoma, placenta | | SEQ. ID NO: 274 | SEQ. ID NO: 275 |
| | (MGC15668) Hypothetical | tumor, lung, colon, stomach carcinomas germ | | | |
| | protein MGC15668 | cell tumors, burkitt lymphoma, | | | |
| Hs.236510 | TPARL TPA regulated locus | Melanoma, rhabdomyosarcoma, renal cell | | SEQ. ID NO: 276 | SEQ. ID NO: 277 |
| | | carcinoma, mucoepidermoid carcinoma, uterus | | | |
| | | carcinoma, B-cell chronic lymphotic | | | |
| | | leukemia, colon carcinoma, lymphoma, ovary | | | |
| | | fibrotheoma, lung carcinoma, kidney tumor, | | | |
| | | breast carcinoma, glioma, parathyroid tumor, | | | |
| | | germ cell tumors, liposarcoma, thyroid | | | |
| | | tumor, lung carcinoid tumors, liposarcoma, | | | |
| | | small intestine duodenal carcinoma, | | | |
| | | genitourinary tract | | | |
| | | transitional cell tumors, head and neck | | | |
| | | carcinoma, melanoma, endometrium carcinoma, | | | |
| | | adrenal cortex carcinoma, osteosarcoma, oral | | | |
| | | carcinoma, synovial sarcoma, lung carcinoma, | | | |
| | | renal cell carcinoma, chondrosarcoma, breast | | | |
| | | carcinoma, melanoma, meningioma, lymphoma, | | | |
| | | chronic myelogenous leukemia, embryonal cell | | | |
| | | carcinoma | | | |
| Hs.356072 | POM73, Moderately similar to | Lung carcinoid tumors, Lung carcinoma | | SEQ. ID NO: 278 | SEQ. ID NO: 279 |
| | POL2_HUMAN RETROVIRUS-RELATED | | | | |
| | POL POLYPROTEIN [H.sapiens] | | | | |
| Hs.336963 | EVX1 Eve, even-skipped homeo | Colon carcinoma | | SEQ. ID NO: 280 | SEQ. ID NO: 281 |
| | box homolog 1 (Drosophila) | | | | |
| Hs.170046 | POM74 | Ovary carcinoma | | SEQ. ID NO: 282 | SEQ. ID NO: 283 |
| Hs.170482 | MYL5 Myosin, light | Ovary tumors, glioma, lung carcinoma, breast | | SEQ. ID NO: 284 | SEQ. ID NO: 285 |
| | polypeptide 5, regulatory | colon and pancreatic carcinoma, kidney | | | |
| | | tumors, leiomyosarcoma, uterus tumors | | | |
| Hs.170993 | POM75 | Kidney tumors, prostatic carcinoma | | SEQ. ID NO: 286 | SEQ. ID NO: 287 |
| Hs.172330 | POM76 | cervical, lung and breast carcinoma, | | SEQ. ID NO: 288 | SEQ. ID NO: 289 |
| | (MGC2705) predicted MGC2705 | retinoblastoma, melanoma, laiomyosarcoma, | | | |
| | | Wilms tumor, breas rhabdomyosarcoma, acute | | | |
| | | myalogenous leukemia, burkitt lymphoma | | | |
| Hs.172603 | POM77 | prostate carcinoma | | SEQ. ID NO: 290 | SEQ. ID NO: 291 |
| Hs.330485 | POM78 | Ovary carcinoma | | SEQ. ID NO: 292 | SEQ. ID NO: 293 |
| Hs.180142 | CLSP Calinodulin-like skin | Skin carcinoma, breast carcinoma, lung | | SEQ. ID NO: 294 | SEQ. ID NO: 295 |
| | protein | carcinoma | | | |
| Hs.328801 | POM79 | Lung carcinoma, breast carcinoma | | SEQ. ID NO: 296 | SEQ. ID NO: 297 |
| Hs.181654 | POM80 | Lung carcinoid tumors, kidney tumors | | SEQ. ID NO: 298 | SEQ. ID NO: 299 |
| Hs.1823E2 | POM90 | ovarian carcinoma, kidney tumors | | SEQ. ID NO: 300 | SEQ. ID NO: 301 |
| Hs.185831 | POM91 | Prostate, stomach and bladder carcinoma | | SEQ. ID NO: 302 | SEQ. ID NO: 303 |
| Hs.189358 | POM92 | lung carcinoid tumors, germ cell tumors, | | SEQ. ID NO: 304 | SEQ. ID NO: 305 |
| | | breast carcinoma | | | |
| Hs.190488 | POM93 | Skin squamous cell carcinoma, stomach | | SEQ. ID NO: 306 | SEQ. ID NO: 307 |
| | (Homo sapiens mRNA; cDNA | carcinoma, colon carcinoma, parathyroid | | | |
| | DKFZpE667M2411 (from clone | tumor, lung carcinoid tumors, glioma, breast | | | |
| | DKFZpE667M2411) | carcinoma, lymphoma, melanoma, uterus | | | |
| | | carcinoma, prostate carcinoma, | | | |
| | | chondrosarcoma, retinoblastoma, cervical | | | |
| | | carcinoma, renal carcinoma, head and neck | | | |
| | | carcinoma, chronic myelogenous leukemia, | | | |
| | | hypernephroma, uterus carcinoma, leiomioma | | | |
| Hs.191574 | POM94 | Pancreas carcinoma, parathyroid tumor, ovary | | SEQ. ID NO: 308 | SEQ. ID NO: 309 |
| | (Homo sapiens cDMA FLJ13050 | tumors, teratocarcinoma, acute myelogenous | | | |
| | fis, clone NT2RP3001432) | leukemia, lung carcinoid tumors, | | | |
| | | hypernephroma, head and neck carcinoma, | | | |
| | | melanoma | | | |
| Hs.193677 | ZNF141 Zinc finger protein | Retinoblastoma, lung carcinoid tumors, | | SEQ. ID NO: 310 | SEQ. ID NO: 311 |
| | 141 (clone pHZ-44) | hypernaphroma, glioma, head and neck | | | |
| | | carcinoma ovary tumors, leiomioma | | | |
| Hs.195081 | POM95 | germ cell tumors | | SEQ. ID NO: 312 | SEQ. ID NO: 313 |
| Hs.195374 | POM96 | germ cell tumors, B-cell chronic lymphotic | | SEQ. ID NO: 314 | SEQ. ID NO: 315 |
| | | leukemia, kidney tumor, uterus tumors | | | |
| Hs.195641 | POM97 | Uterus carcinoma, Lung carcinoma, colon | | SEQ. ID NO: 316 | SEQ. ID NO: 317 |
| | | carcinoma, nervous cell tumors, breast | | | |
| | | carcinoma, stomach carcinoma | | | |
| Ha.196073 | POM98 | Lung carcinoma, germ cell tumors, stomach | | SEQ. ID NO: 318 | SEQ. ID NO: 319 |
| | | carcinoma, genitourinary tract transitional | | | |
| | | cell carcinoma | | | |
| Hs.199460 | DPCR1 DPCR1 protein | Pancreas carcinoma, stomach carcinoma | | SEQ. ID NO: 320 | SEQ. ID NO: 321 |
| Hs.202247 | POM99 | lung carcinoid tumors | | SEQ. ID NO: 322 | SEQ. ID NO: 323 |
| Hs.202512 | POM100 | lung carcinoid tumors, colon carcinoma | | SEQ. ID NO: 324 | SEQ. ID NO: 325 |
| Hs.202577 | POM101 (Homo sapiens cDNA | Schwannoma, lung carcinoid tumors, germ cell | | SEQ. ID NO: 326 | SEQ. ID NO: 327 |
| | FLJ12166 fis, clone | tumors, lymphoma, colon carcinoma, glioma | | | |
| | MAMMA1000616) | | | | |
| Hs.202612 | POM102 | Lung carcinoma, colon carcinoma | | SEQ. ID NO: 328 | SEQ. ID NO: 329 |
| Hs.209560 | POM103 | Lung carcinoma, embryonal cell carcinoma, | | SEQ. ID NO: 330 | SEQ. ID NO: 331 |
| | | pituitary tumor | | | |
| Hs.209646 | POM104 | Lung carcinoma, choriocarcinoma, melanoma | | SEQ. ID NO: 332 | SEQ. ID NO: 333 |
| | (KIAA1118) | glioblastoma, neuroblastoma, osteosarcoma, | | | |
| | KIAA1118 protein | colon carcinoma, breast carcinoma, lymphoma, | | | |
| | | glioma, retinoblastoma | | | |
| Hs.211238 | IL-1H1 Interleukin-1 homolog | colon carcinoma, head and neck carcinoma | | SEQ. ID NO: 334 | SEQ. ID NO: 335 |
| | 1 | | | | |
| Ha.217766 | POM105 | Ovary carcinoma | | SEQ. ID NO: 336 | SEQ. ID NO: 337 |
| Hs.217882 | POM106 | glioma, colon carcinoma, kidney tumors, | | SEQ. ID NO: 338 | SEQ. ID NO: 339 |
| | | prostate tumors, lung | | | |
| | | carcinoma, hypernephroma, head and neck | | | |
| | | carcinoma, duodenal carcinoma, melanoma, | | | |
| | | pancreatic carcinoma, uterus tumors | | | |
| Hs.220529 | CEACAM5 Carcinoembryonic | Pancreas carcinoma, colon carcinoma, stomach | KNOWN TUMOR | SEQ. ID NO: 340 | SEQ. ID NO: 341 |
| | antigen-related cell adhesion | carcinoma, head and neck carcinoma, lung | MARKER | | |
| | molecule 5 | carcinoma leiomioma, breast carcinoma | | | |
| Hs.222056 | POM107 Homo sapiens cDNA | Stomach carcinoma, head and neck | | SEQ. ID NO: 342 | SEQ. ID NO: 343 |
| | FLJ11572 fis, clone | carcinoma, breast carcinoma | | | |
| | HEMBA1003373 | | | | |
| Hs.225083 | POM108 | Melanoma, ovary tumors, colon carcinoma, | | SEQ. ID NO: 344 | SEQ. ID NO: 345 |
| | | parathyroid tumor, kidney tumors, head and | | | |
| | | neck carcinoma | | | |
| Hs.227098 | GCMB Glial cells missing | perathyroid_tumor | | SEQ. ID NO: 346 | SEQ. ID NO: 347 |
| | homolog b (Drosophila) | | | | |
| Hs.239107 | POM109 | Lymphoma, germ cell tumors, head and neck | | SEQ. ID NO: 348 | SEQ. ID NO: 349 |
| | | carcinoma | | | |
| Hs.239891 | GPR35 G protein-coupled | B-cell chronic lymphocytic leukemia, colon | SURFACE | SEQ. ID NO: 350 | SEQ. ID NO: 351 |
| | receptor 35 | carcinoma, pancreas and carcinoma | | | |
| HS.241381 | CRSP7 Cofactor required for | Pancreatic carcinoma, duodenal carcinoma, | | SEQ. ID NO: 352 | SEQ. ID NO: 353 |
| | Sp1 transcriptional | ovary carcinoma, melanoma, osteosarcoma, | | | |
| | activation, subunit 7 (70kD) | glioma, leiomyosarcoma, germ cell tumors | | | |
| Hs.241407 | SERPINB13 Serine (or | ORAL carcionoma, cervical carcinoma, head | | SEQ. ID NO: 354 | SEQ. ID NO: 355 |
| | cysteine) proteinase | and neck carcinoma | | | |
| | inhibitor, clade B | | | | |
| | (ovalbumin), member 13 | | | | |
| Hs.243920 | POM110 | Pancreas carcinoma | | SEQ. ID NO: 356 | SEQ. ID NO: 357 |
| Hs.244378 | SLC2A6 Solute carrier family | Hypernephroma, pancreatic carcinoma, gliona, | | SEQ. ID NO: 358 | SEQ. ID NO: 359 |
| | 2 (facilitated glucose | lung carcinoma, neuroblastoma, renal cell | | | |
| | transporter), member 6 | carcinoma, adrenal gland tumors | | | |
| Hs.246781 | POM111 | parathyroid_tumor, lung carcinoid tumors, | | SEQ. ID NO: 360 | SEQ. ID NO: 361 |
| | | germ cell tumors, hepatocellular carcinoma, | | | |
| | | stomach carcinoma, breast carcinoma | | | |
| Hs.247817 | H2B/S Histone family member A | Breast carcinoma, chronic myelogerious | | SEQ. ID NO: 362 | SEQ. ID NO: 363 |
| | | leukemia, cervical carcinoma, melanoma, | | | |
| | | ovary carcinoma, lung carcinoma, | | | |
| | | osteosarcoma, mucoepidermoid carcinoma, | | | |
| | | duodenal carcinoma, leiomyosarcoma, glioma, | | | |
| | | prostate carcinoma, kidney tumors, colon | | | |
| | | carcinoma, prostatic intraepithelial | | | |
| | | neoplasia, lymphoma, uterus carcinoma, | | | |
| | | parathyroid tumor, insulinoma, | | | |
| | | chondrosarcoma, ovary tumors, multiple | | | |
| | | myeloma, chondrosarcoma, bladder tumors, | | | |
| | | parathyroid tumors, insulinoma, breast | | | |
| | | carcinoma, pnet tumors, | | | |
| Hs.250158 | POM112 | Head and neck carcinoma, stomach carcinoma, | | SEQ. ID NO: 364 | SEQ. ID NO: 365 |
| | | colon carcinoma | | | |
| Hs.250848 | Pom113Homo sapiens cDNA | Uterus carcinoma, prostate tumor, glioma, | | SEQ. ID NO: 366 | SEQ. ID NO: 367 |
| | FLJ14761 fis, clone | duodenal carcinoma, colon carcinoma, glioma, | | | |
| | NT2RP3003302 | stomach carcinoma, Germ cell tumors, lung | | | |
| | | carcinoma, embryonal cell carcinoma, breast | | | |
| | | carcinoma, choriocarcinoma | | | |
| Hs.252351 | HHLA2 HERV-H LTR-associating | Colon carcinoma, kidney tumors, ovary | | SEQ. ID NO: 368 | SEQ. ID NO: 369 |
| | 2 | tumors, Stomach tumors, prostate carcinoma, | | | |
| Hs.253298 | POM114 | Head and neck carcinoma, germ cell tumors | | SEQ. ID NO: 370 | SEQ. ID NO: 371 |
| Hs.254379 | POM115 | Ovary carcinoma | | SEQ. ID NO: 372 | SEQ. ID NO: 373 |
| Hs.255877 | POM116 | Leukemia | | SEQ. ID NO: 374 | SEQ. ID NO: 375 |
| Hs.266390 | POM117 | Lung carcinoid tumors, pre-B cell acute | | SEQ. ID NO: 376 | SEQ. ID NO: 377 |
| | | lymphoblastic leukemia, ovarian carcinoma | | | |
| Hs.268171 | POM118 | Nervous cell tumors, germ cell tumors, | | SEQ. ID NO: 378 | SEQ. ID NO: 379 |
| | | prostatic intraepithelial neoplasia, ovary | | | |
| | | tumors | | | |
| Hs. 106823 | STX12 Syntaxin 12 | Bladder carcinoma, colon carcinoma, | | SEQ. ID NO: 380 | SEQ. ID NO: 381 |
| | and | lymphoma, prostate carcinoma, pancreas | | SEQ. ID NO: 382 | SEQ. ID NO: 383 |
| | MGC14797 Flypothetical protein | carcinoma, breast carcinoma, Wilms' tumor | | | |
| | MGC14797 | uterus carcinoma, meningioma, kidney | | | |
| | | tumors, lung carcinoma, stomach carcinoma | | | |
| | | parathyroid tumor, germ cell tumors, ovary | | | |
| | | tumors, B-cell chronic lymphocytic leukemia, | | | |
| | | germ cell tumors, thyroid tumor, | | | |
| | | leiomyosarcoma, duodenal carcinoma, | | | |
| | | pancreatic carcinoma, alveolar | | | |
| | | rhabdomyosarcoma, glioma, head and neck | | | |
| | | carcinoma, bladder transitional cell | | | |
| | | papilloma, retinoblastoma, chondrosarcoma | | | |
| | | Stomach carcinoma, pre-B call acute | | | |
| | | lymphoblastic leukemia, lung carcinoma, | | | |
| | | hepetocellular carcinoma, melanoma, | | | |
| | | fibrosarcoma, lymphoma, chondrosarcoma, | | | |
| | | osteosarcoma, hepatocellular carcinoma, | | | |
| | | burkitt lymphoma, uterus carcinoma | | | |
| Hs.355428 | Pom119, Weakly similar to | Pancreas carcinoma, glioma, breast | | SEQ. ID NO: 384 | SEQ. ID NO: 385 |
| | B34087 Predicted protein | carcinoma, lung carcinoid tumors, Ewing's | | | |
| | [H.sapiens] | sarcoma, colon carcinoma, melanoma, lung | | | |
| | | carcinoma, head and neck carcinomar, ovary | | | |
| | | carcinoma, pnet tumor | | | |
| Hs.272216 | GPE Glycoprotein VI | Rhabdomyosarcoma, colon carcinoma, head and | | SEQ. ID NO: 386 | SEQ. ID NO: 387 |
| | (platelet) | neck carcionoma, epidydimal tumors, nervous | | | |
| | | cell tumors | | | |
| Hs.272499 | DHRS2 Dehydroganase/reductase | Bladder transitional cell papilloma, | | SEQ. ID NO: 388 | SEQ. ID NO: 389 |
| | (SDR family) member 2 | melanoma, colon carcinoma, hepatocellular | | | |
| | | carcinoma, endometrial carcinoma, lung | | | |
| | | carcinoid tumors, colon carcinoma, | | | |
| | | Lymphoma, fibrosarcoma, kidney_tumor, | | | |
| | | meningioma, genitourinary tract transitional | | | |
| | | cell tumors, fibrosarcoma, Stomach tumor, | | | |
| | | breast carcinoma, | | | |
| Hs.273625 | POM120 | Stomach carcinoma | | SEQ. ID NO: 390 | SEQ. ID NO: 391 |
| Hs.278291 | POM121 Weakly similar to | endometrial carcinoma | | SEQ. ID NO: 392 | SEQ. ID NO: 393 |
| | 810024J URF 4 [H.sapiens] | | | | |
| Hs.279805 | POM122 | Lung carcinoid tumors, nervous cell tumors, | | SEQ. ID NO: 394 | |
| | | pnet tumor, | | | |
| Hs.280146 | POM123 Weakly similar to L1 | Lung carcinoid and ovarian tumorsm glioma | | SEQ. ID NO: 395 | |
| | repeat, Tf subfamily, member | | | | |
| | 18 [Mus musculus] | | | | |
| Hs.109274 | Pom124 | Lung carcinoma, stomach carcinoma, colon | | SEQ. ID NO: 396 | SEQ. ID NO: 397 |
| | MGC4365 Predicted protein | carcinoma, breast carcinoma, glioma, kidney | | | |
| | MGC4365 | tumors, melanoma, choriocarcinoma, t- cell | | | |
| | | leukemia, cervical carcinoma, | | | |
| | | neuroblastoma, retinoblastoma, multiple | | | |
| | | myeloma, ovary carcinoma, pre-B cell acute | | | |
| | | lymphoblastic leukemia, uterus carcinoma, | | | |
| | | kidney tumors, lung carcinoma, endometrial | | | |
| | | carcinoma, renal cell carcinoma, acute | | | |
| | | myelogenous leukemia cell, cervical carcinoma | | | |
| Hs.282050 | POM125 | Prostate carcinoma, embryonal cell | | SEQ. ID NO: 398 | SEQ. ID NO: 399 |
| | Homo sapiens cDNA FLJ31265 | carcinoma, ovary carcinoma, kidney tumors, | | | |
| | fis, clone KIDNEY2006030, | colon carcinoma, germ cell tumors, | | | |
| | moderately similar to Gallus | neuroblastoma, retinoblastoma, melanoma, | | | |
| | gallus syndesmos mRNA | breast carcinoma, ovary tumors, renal cell | | | |
| | | carcinoma, endometrium carcinoma, | | | |
| | | leiomyosarcoma, glioma, head and neck | | | |
| | | carcinoma, nervous cell tumors, | | | |
| | | neuroblastoma, cervical carcinoma, leukemia, | | | |
| | | ovarian carcinoma, head and neck tumors, | | | |
| Hs.284203 | MYOD1 Myogenic factor 3 | Rhabdomyosarcoma, burkitt lymphoma | | SEQ. ID NO: 400 | SEQ. ID NO: 401 |
| Es.285026 | HHLA1 HERV-H LTR-associating | Colon carcinoma | | SEQ. ID NO: 402 | SEQ. ID NO: 403 |
| | 1 | | | | |
| Hs.285887 | POM12E Weakly similar to | hepatocellular carcinoma | | SEQ. ID NO: 404 | SEQ. ID NO: 405 |
| | 2109260A B cell growth factor | | | | |
| | [H. sapiens] | | | | |
| Hs.285894 | POM127 | hepatocellular carcinoma | | SEQ. ID NO: 406 | SEQ. ID NO: 407 |
| Hs.288568 | POM128 | Stomach carcinoma | | SEQ. ID NO: 408 | SEQ. ID NO: 409 |
| | FLJ22644 Predicted | | | | |
| | protein FLJ22644 | | | | |
| Hs.288842 | OPA3 Optic atrophy 3 | Lymphoma, kidney renal cell carcinoma, lung | | SEQ. ID NO: 410 | SEQ. ID NO: 411 |
| | (autosomal recessive, with | small cell carcinoma, pancreas carcinoma, | | | |
| | chorea and spastic | choriocarcinoma, | | | |
| | paraplegia) | Melanoma, retinoblastoma, leiomyosarcoma, | | | |
| | | prostate carcinoma, head and neck carcinoma, | | | |
| | | parathyroid tumor, choriocarcinoma | | | |
| Hs.290308 | POM129 | ovarian carcinoma, glioma, hepatocellular | | SEQ. ID NO: 412 | SEQ. ID NO: 413 |
| | | carcinoma, breast carcinoma, head and neck | | | |
| | | carcinoma, insulinoma, retinoblastoma | | | |
| Hs.293678 | TCBAP075B Predicted | Retinoblastoma, leiomyosarcoma, lymphoma, | | SEQ. ID NO: 414 | SEQ. ID NO: 415 |
| | protein TCBAP0758 | neuroblastoma, glioma, cervical carcinoma, | | | |
| | | pancreas carcinoma, germ cell tumors, | | | |
| | | stomach carcinoma, glioma, uterus carcinoma, | | | |
| | | lung carcinoid tumors, adrenal cortex | | | |
| | | carcinoma, ovary tumors, melanoma, | | | |
| | | lymphoblastic leukemia, colon cancer, | | | |
| | | endometrial carcinoma, neuroblastoma, breast | | | |
| | | carcinoma, head and neck neck carcinoma, | | | |
| | | nervous cell tumors, lung carcinoma, Wilms' | | | |
| | | tumor, pancreas carcinoma | | | |

Of the tumor associated EST's detected by the methods of the present invention, a particularly interesting group are the clusters represented by EST's found exclusively in tumor derived libraries. One striking feature of these tumor markers is their frequent occurrence in colon, lung and ovarian carcinomas. Thus, the high percentage of tumor-specific EST's is characteristic of highly malignant tumors (e.g. ovary carcinomas, metastatic breast carcinomas and small cell lung tumors. Accordingly, the methods of the present invention provide a method for predicting malignancy of a tumor based on the percentage of tumor-specific EST expression detected in such tumors. Utilizing standard molecular biology techniques as exemplified below, for example, persons of ordinary skill in the art can utilize probes for tumor associated EST's to determine the level of malignancy in a tumor tissue sample.

All three colon-specific clusters detected with the methods of the present invention represented known genes which encode apolipoprotein B mRNA editing protein APOBEC1, guanylate cyclase 2C and G protein coupled receptor 35. Both APOBEC1 and guanylate cyclase 2C mRNAs have been shown to be overexpressed in colon carcinomas (Lee et al, Gastroenterology 115(5):1096-1103 (1998); Carithers et al. Proc.Natl. Acad. Sci. USA 93(25):14827-32 (1996). Moreover, high level expression of APOBEC1 in transgenic mice and rabbit livers causes liver dysplasia and hepatocellular carcinomas and guanylate cyclase 2C appears to be relatively specific marker for the presence of metastatic colonic carcinoma cells. These observations, together with the appearance of the guanylate cyclase 2C in tumor specific clusters, indicate that this gene is a putative marker of progression of colon cancer.

### EXAMPLE 2

In order to detect the presence of a tumor associated EST in actual tissue samples, biological samples were prepared and analyzed for the presence or absence of the EST sequence. In each case, where clusters are defined by a plurality of sequences, the probes utilized are derived from the longest reported sequence for the cluster. Individual subsets of EST clusters predicted to be tumor associated with the methods of the present invention were analyzed in polymerase chain reaction studies on Clontech multiple tissues cDNA (MTC) panels and on panels of genomic DNA from different animal species. Gene or gene fragments corresponding to EST clusters Hs.133107, Hs.154173 and Hs.67624 according to our computational differential display studies were expressed only in tumors. Hs.133244 was expressed in a variety of tumors and was also expressed at very low levels in normal testis and germinal B-cells. Initially, the screening method involved a non-PCR based strategy. Such screening methods include two-step label amplification methodologies that are well known by persons of ordinary skill in the art. Both PCR and non-PCR based screening strategies can also detect target sequences with a high level of sensitivity.

A subset of EST clusters found by HSAnalyst software was analyzed by both confirmatory PCR on Clontech Multiple Tissue cDNA Panels. PCR Amplification of the tumor associated EST Hs.133294 Fragment was analyzed in Human Tumor MTC Panel 1 and 2, Human Immune System MTC Panel, Human Fetal MTC Panel, DNA from Different Animal species, and Southern hybridization of Hs.133294 fragment with genomic DNA from different animal species digested to completion with EcoR I. Hs.133294 represents an EST protein-encoding mRNA located on chromosome 1q21. It is weakly similar in homology to IQGA (human RAS GTPase-activating-like protein IQGAP1). Hs.133294 was represented in: prostate tumor, HNSCC, breast carcinoma, oligodendroglioma, colon carcinoma, CML, lung carcinoma, ovarian carcinoma, uterus carcinoma, adrenal adenoma and <<minor occurrences>> in normal testis and germinal B-cells. One EST in the cluster was derived from normal testis, one from germinal B-cells and twenty-five from different tumors. Both testis and germinal B-cells as tissues are known to express tumor markers, e.g. cancer-testis antigen family members are expressed only in testis in a healthy organism, but testis expression does not interfere with the tumor marker features of such a genes. Unlike in the case of the other examples contained herein, where primers were selected from the same exon, in this case primers belong to two different exons separated by intron 672 bp in size. That is why two fragments may be considered as specific to Hs.133294: a 1084 bp fragment which corresponds to unspliced mRNA and a 412 bp fragment corresponding to spliced mRNA. PCR on human tumor MTC panel produced the 1084 bp fragment on cDNAs from all eight tumors comprising the panel. The 412 bp fragment was not generated in samples from prostatic adenocarcinoma, lung carcinoma and colon adenocarcinoma propagated as xenografts in athymic nude mice. The 412 bp fragment was generated in lung carcinoma and colon adenocarcinoma which have been taken as surgical explants from metastasis and primary tumor. PCR of cDNA from testis generated the 412 bp fragment detected in normal human MTC panels 1 and 2 and weak detection of the 1084 bp fragment. No fragments were produced on human immune system MTC panel. But on human fetal MTC panel both 1084 bp and 412 bp fragments were amplified in cDNAs from all organs and/or tissues represented in the panel. One thousand eighty four base pairs fragment corresponding to unspliced mRNA was detected in all lanes in relatively greater amounts than the 412 bp fragment. The weakest signals for both fragments were detected for fetal brain and heart.

### EXAMPLE 3

Utilizing similar methods as in Example 2, Hs.154173, a non-coding mRNA with tumor expression located in the intergenic spacer region within the rRNA encoding unit and is represented in lung carcinoma and testicular teratocarcinoma was analyzed for expression in the various tissue panels as in Example 2. PCR testing with Hs.154173 specific primers on human tumor MTC panel resulted in amplification of an Hs.154173-specific fragment of 443 bp in the lanes corresponding to breast carcinoma and pancreatic adenocarcinoma. There was also a weak band in the lane that corresponded to prostatic adenocarcinoma.

In contrast, PCR analysis with the same Hs.154173-specific primers on normal human MTC panels 1 and 2, on human immune system MTC panel and human fetal MTC panel demonstrated no amplification of the corresponding fragment in any of 31 normal tissues cDNA comprising these four normal panels, indicating that this fragment is not expressed in these tissues.

### EXAMPLE 4

Hs.67624 is a tumor-associated non coding mRNA located on Chromosome 3 and represented in germ cell tumors and head and neck squamous cell carcinoma. The results of PCR amplification of the tumor associated EST Hs.67624 fragment in Human Tumor MTC Panel 1 and 2, Human Immune System MTC Panel, Human Fetal MTC Panel, DNA from different animal species, and Southern hybridization of Hs.67624 fragment with genomic DNA from different animal species on genomic DNA digested to completion with EcoRI. These results confirmed that HS 67624 as a tumor associated EST expressed in ovarian carcinoma. There are three human tissues that often express tumor antigens. These are thymus, testis and embryonic tissues. PCR with Hs.67624-specific primers on human tumor MTC panel resulted in predicted amplification of 315 bp Hs.67624-specific fragment in ovarian carcinoma. PCR with the same Hs.67624 primers on normal human MTC panels 1 and 2 resulted in no fragments on any of 16 normal cDNA libraries comprising these panels. PCR on human immune system MTC panel and human fetal MTC panel produced signals corresponding to 315 bp fragment only on cDNA from thymus. The signal in fetal thymus was considerably stronger than for normal thymus.

### EXAMPLE 5

Hs.133107 is a tumor associated non-coding mRNA located on chromosome 12p13. The results of PCR Amplification of the EST Hs.133107 fragment in Human Tumor MTC Panel 1 and 2, Human Immune System MTC Panel, Human Fetal MTC Panel. These results confirmed that Hs.133107 as a tumor related EST. PCR on normal Human MTC Panels 1 and 2 produced no fragments on any of cDNA from 16 normal tissues. PCR on human immune system MTC panel resulted in amplification of 344 bp fragment on cDNA from lymph node. PCR on human fetal MTC panel did not result in any fragments.

### EXAMPLE 6

The results of PCR Amplification of the a nucleic acid specific for Glucose 3 phosphate dehydrogenase fragment in Human Tumor MTC Panel 1 and 2, Human Immune System MTC Panel, Human Fetal MTC Panel and DNA from different animal species was performed as in the above examples. This control demonstrated that mRNA specific for Glucose 3 phosphate dehydrogenase could be detected in a manner consistent with known expression patterns of this gene.

### EXAMPLE 7

The methods of the present invention were used to detect differential expression of genes expressed in hyperosmotic stress (caused by NaCl), or dehydration in the plant Arabidopsis thaliana. Despite the relatively small number of ESTs and UNIGENE clusters available for this organism, 5 stress-associated clusters were detected using the methods of the present invention. Three stress-associated clusters detected in A. thaliana represented known plant genes involved in stress response: GST30, Lti30 and cor15-encoding gene. The remaining clusters represented unknown genes. The applicability of the methods of the present invention to A. thaliana provides a prognostic model useful to determine if the relevant genes found in A. thaliana can be used as a hybridization templates to find orthologs in other agricultural plants and such orthologs will be useful for gene targeting etc in such important plants.

Utilizing the methods of the present invention, a database “AT Lib Registry” was constructed. This database contained descriptions of all cDNA expression libraries used to build an EST database for A. thaliana. Computer-based methods were used to determine mRNA sequences differentially expressed in plants under different physiological conditions including oxidative, herbicidal and other stress types. The CDD permitted an analysis of the absolute number of nucleotide sequences synthesized for transcription matrices of every type of interest in discovered samples. The CDD analysis utilized data from databases such as dbEST containing more than 110 000 EST sequences that were deduced from cDNA libraries made from A. thaliana cells. For every sequence in the database there was a description of source cDNA library provided. These data and the EST clustering information complete the dataset needed to describe a tissue-associated (or condition-associated) expression of transcripts of every type (or genes). The processing of large volumes of EST information was facilitated by means of a variation of the Hs.Analyst software utilized for determination of tumor-associated markers wherein the variation utilized the Hs.Analyst main module and an Arabidopsis LibRegistry, dividing the Arabodopsis.libraries according to stress/non-stress categories.

The software At_Analyst was utilized to analyze EST clustering data of the model plant Arabidopsis thaliana and to conduct a comparative analysis of gene expression spectra in different tissues of the plant. In this example, all data sources were divided into 3 classes named “target1”, “target2” and “undefined”, whereas the last class pooled data were not entered in either of first two classes.

At_Analyst software description. In this example, the source data for the program were arranged in two plain text files designated “at.data” and “libraries”. The file “at.data” contained cluster descriptions arranged according to individual clusters. All fields were listed each in a separate line for each EST. Each cluster description with a field “ID” which contained the internal UniGene cluster index, the cluster gene “title” and gene name if there was significant known homology of a cluster to a known gene, the number of sequences of any type (mRNA, protein, cDNA) included in cluster and lines containing information about all individual sequences of the cluster. For each sequence there was provided a LID (Library ID) which data field was LID used to retrieve information about the EST source library, thereby allowing association of the EST sequence with a particular physiological state or growth condition.

The database “At Library Registry” was created. This database included all source cDNA clone library descriptions of 71 libraries prepared from different parts or tissues of A. thaliana. Every record consisted of the following fields: 1) library ID in dbEST database; 2) library name; 3) tissue source of mRNA used to prepare cDNA sequences and additional comments concerning library construction methods and physiological conditions of plant growth; 4) organism name (A. thaliana in the present example); 5) organism strain or ecotype; and 6) cloning vector used for library construction. In general, source tissues were derived from A. thaliana strains Columbia Col-0, Columbia C24, Columbia GH50, Columbia g11, Landsberg erecta and Ohio State. Some of the libraries in the database were obtained from plant parts like aboveground organs, roots, flower buds, green siliques, immature siliques, inflorescence, rosettes, seedling hypocotyls and some from different specific cell types. There were also included a number of clone libraries made from cultured cell lines of A.thaliana.

All clone libraries in the At Library Registry were separated into four general types: 1) “untreated” indicated clone libraries made from normal plants and its parts cultivated under normal conditions; 2) “treated”—indicated libraries made from plants subjected to any kind of stressing; 3) “low-level” indicated clone libraries prepared from genomic DNA, not on mRNA; 4) “undefined”—indicated clone libraries whose origin could not be deduced with the available information. The resulting base AT Library Registry was presented by a Microsoft Excel workbook consisting of four worksheets, one for each type of clone library class as mentioned above. The total number of sequences that were derived from clone libraries included in AT Library Registry was 113 023 ESTs.

A round of CDD was conducted when we found quantitative percentages of transcription pools volumes of plants exposed to stress conditions and plants grew in normal physiological conditions. Statistical analysis of expression spectra has revealed the quantitatively reliable differences among plants exposed to salt (hyperosmotic) stresses. The results are presented in Table 3. The conditions for comparing the clusters compared EST's from stress-induced Arabidopsis to normal plants contained EST's expressed in stress-exposed plants. Genes (clusters) of interest demonstrated to be associated with Arabidopsis stress conditions were At.11290 (glutathione S-transferase), At.5388 (Iti30) and At.20845 (COR15 polypeptide).

TABLE 3
Sequences of clusters differentially expressed under salt stress conditions.

| | | | Protein | | |
|---|---|---|---|---|---|
| Cluster | | All | sequences | Target | Background |
| ID | Gene presented by cluster{tc \\| 2″″} | sequences | {tc \\| 1″″} | sequences | sequences |
| At.5801 | Arabidopsis thaliana AT3g28220/T19D11_3 mRNA, | 10 | 2 | 7 | 1 |
| | complete cds | | | | |
| At.5388 | Arabidopsis thaliana (Landsberg Erecta) lti30 mRNA | 13 | 3 | 8 | 1 |
| At.11290 | Arabidopsis thaliana chromosome I glutathione S-transferase | 13 | 3 | 8 | 2 |
| | (GST30) mRNA, complete cds | | | | |
| At.12464 | Arabidopsis thaliana chromosome II section 206 of | 13 | 1 | 11 | 1 |
| | 255 of the complete sequence. Sequence from clones F16M14 | | | | |
| At.20845 | Arabidopsis thaliana mRNA for COR15 polypeptide | 32 | 4 | 24 | 4 |

The methods of the present invention are also applicable to other agricultural plants that are well represented in the UniGene database. For example, as of Nov. 20, 2001, there were 34812 sequences in 4012 clusters for Hordeum vulgare, 47841 sequences in 12836 clusters for Oryza sativa, 31826 sequences in 2744 clusters for Triticum aestivum and 69231 sequences in 7171 clusters for Zea mays. Furthermore, the methods of the present invention may be applied to other organisms additional datasets are developed that build clusters similar to UniGene database. There are 208198 sequences available for Glycine max, 141687 sequences for Lycopersicon esculentum, 137588 sequences for Medicago truncatula, 76645 sequences for Sorghum bicolor and 55637 sequences for Solanum tuberosum. Since about 113 000 sequences were enough to obtain statistically reliable results in our investigation it is reasonable to recommend using of CDD method for searching for stress-induced genes in the above mentioned plants as done with Arabidopsis.

The investigation of Arabidopsis thaliana associated ESTs derived from clone libraries made from the stress-exposed and normal plants revealed three genes that encoded proteins that were overexpressed-in-stress proteins (as used herein, the term “stress-overexpressed applies to the fact that 80% or more of the sequences from their clusters are derived from plant grown in stress conditions. The available clone libraries were also adequate for investigation of salt-induced stress. Thus, seven of eight total ESTs in cluster AT.5801 were derived from library m27 made from 10-14-days old shoots treated by 160 mM NaCl solution for several hours. Eight of a total of nine ESTs of cluster At. 11290 are also derived from this clone library. Cluster At.20845 consists of 22 ESTs from the same clone library 27, 2 ESTs from the plant parts treated by 200 mM NaCl (library numbers 15 and 40) and 4 ESTs from the parts of normal plant. Library 27 was deliberately enriched by sequences specifically expressed in salt stressed plant whereas libraries 15 and 40 were not as can bee seen quite clearly from the typical stress-induced cluster structures (as e.g., At.20845). It is clear also that the CDD methods of the present invention are more productive than an experimental approach which is not sensitive enough to distinguish between low levels of expression of salt-induced genes.

One of the revealed clusters At.11290 represented the glutathione-S-transferase gene (GST30). It is known that glutathione transferases are involved in different stress-induced pathways. For example the expression of one of these transferases is increasing the plant's resistance for the aluminum abundance. Moreover, it was shown that such plants are display a significant increase of oxidative stress resistance which can be seen when straining the plant's roots with H(2)DCFDA (Ezalki B. et al., 2001 Plant Physiol November 2001;127(3):918-927). It is also known that the induction of glutathione-S-transferases occurs when the plant is infected with Peronospora parasitica or Pseudomonas syringae pv. Tomato, when the plant is treated by some kind of herbicides and even when the leaf structure is broken (Rairdan G J et al., 2001 Mol Plant Microbe Interact October 2001;14(10):1235-46; Vollenweider S et al., 2000 Plant J November 2000;24(4):467-76). The level of glutathione-S-transferase gene also increases when the plant cells are treated with auxine, salicylic acid or hydrogenic peroxide (Chen W. Singh KB 1999 Plant Physiol November 2001;127(3):918-927). As it can be deduced from published data the glutathione-S-transferase gene is often overexpressed under different kinds of stress conditions in plants. Nevertheless as it is shown in our work, this gene is specifically expressed under salt stress conditions and may serve as marker for this kind of stress.

The other revealed cluster At.5388 represents the gene 1ti30 coding dehydrine 1ti30 which synthesis is induced under the low-temperature stress but not in plants treated by abscizic acid or drought or cold (Welin B. V. et al., 1994 Plant Mol Biol October 1994;26(1):131-44). The cluster At.20845 is representing cor15 protein which shows even more cryoprotective activity than BSA or sacharose (Lin C, Thomashow M F, 1992 Biochem Biophys Res Commun March 31, 1992; 183(3):1103-8). So far as both genes were revealed in our CDD experiments with salt stress-induced genes it might be reasonable to suppose a common underlying processes of regulation of the salt- and temperature-induced plant response.

What is claimed is:

1. A method for determining whether a nucleic acid is a marker for a predetermined phenotype or cell type of interest from a biological species which comprises:
(a) providing a database of expressed sequence tag sequences (EST's) from the species;
(b) placing said EST's in groups termed clusters based on homology of EST's within each cluster;
(c) determining for each cluster the total number of EST's within said cluster;
(d) ordering said clusters sequentially based on the number of EST's in each cluster;
(e) dividing said ordered clusters into subranges based on the number of EST's per cluster;
(f) determining for each cluster subrange obtained from step (e) the number EST's within said cluster which are expressed in said predetermined cell type of interest;
(g) calculating according to a normal distribution the number of clusters in each subrange expected to contain a predetermined threshold percentage of EST's expressed in said cell type of interest, wherein said threshold percentage is a percentage from about 10% to about 100%;
(h) determining the number of clusters in each subrange observed to contain said predetermined threshold percentage of EST's expressed in said predetermined cell type; and
(i) identifying subranges having an observed number of clusters that meet said predetermined threshold percentage greater than the number of clusters expected to meet said predetermined threshold percentage for the subrange according to normal distribution;
wherein if the percentage of EST's expressed in said cell type of interest in a cluster identified in (i) is equal to or greater than said predetermined threshold percentage, said cluster contains a nucleic acid that is a marker for the cell type of interest.

2. The method of claim 1 wherein one or more of the steps are performed on a computer.

3. The method of claim 1 wherein the individual clusters are divided into subranges exponentially.

4. The method of claim 1 wherein the individual clusters are divided into subranges linearly.

5. The method of claim 1 wherein the predetermined threshold percentage of EST's expressed in said cell type of interest is a percentage of about 50% to 100%.

6. The method of claim 1 wherein the predetermined threshold percentage of EST's expressed in said cell type of interest is a percentage of about 70% to 100%.

7. The method of claim 1 wherein the predetermined threshold percentage of EST's expressed in said cell type of interest is a percentage of about 80% to 100%.

8. The method of claim 1 wherein the predetermined threshold percentage of EST's expressed in said cell type of interest is a percentage of about 90% to 100%.

9. The method of claim 1 wherein the predetermined threshold percentage of EST's expressed in said cell type of interest is a percentage of at least 80%.

10. The method of claim 1 wherein the predetermined threshold percentage of EST's expressed in said cell type of interest is a percentage of at least 90%.

11. The method of claim 1 wherein the predetermined threshold percentage of EST's expressed in said cell type of interest is a percentage of at least 95%.

12. The method of claim 1 wherein the predetermined threshold percentage of EST's expressed in said cell type of interest is a percentage of 100%.

13. A method as in claim 1 wherein the cell type of interest is an abnormal cell.

14. The method of claim 1 or claim 13 wherein step (i) comprises identifying subranges having an observed number of clusters meeting said predetermined threshold percentage at least five times greater than the number expected for the subrange according to normal distribution.

15. The method of claim 1 or claim 13 wherein step (i) comprises identifying subranges having an observed number of clusters meeting said predetermined threshold percentage at least one standard deviation greater than the number expected for the subrange according to normal distribution.

16. The method of claim 1 or claim 13 wherein the species is human.

17. The method of claim 16 wherein the individual clusters are divided into subranges exponentially.

18. The method of claim 16 wherein the individual clusters are divided into subranges exponentially.

19. The method of claim 16 wherein the predetermined threshold percentage of EST's expressed in a tumor cell is at least 90%.

20. The method of claim 16 wherein the predetermined threshold percentage of EST's expressed in a tumor cell is 95%.

21. The method of claim 16 wherein the predetermined threshold percentage of EST's expressed in a tumor cell is 100%.

22. A method for determining the progression of colon cancer in a human which comprises determining the level of expression of guanylate cyclase 2C in a cell, wherein if the level of guanylate cyclase 2C expression is greater than the level of expression of guanylate cyclase 2C in normal cells, said cell is a tumor cell.

23. The method of claim 22 wherein the level of the guanylate cyclase 2C is detected by determining the level of mRNA expression for the guanylate cyclase 2C gene.

24. An isolated antibody which specifically binds to a tumor-associated antigen encoded by a nucleic acid selected from the group consisting of SEQ IDNO:'s 9, 11, 13, 15, 17, 19, 23, 25, 27, 29, 33, 35, 37, 39, 41, 45, 47, 55, 57, 59, 61, 63, 65, 67, 69, 73, 75, 77, 79, 81, 83, 89, 91, 93, 95, 97, 99, 101, 103, 107, 109, 111, 113, 115, 117, 119, 121, 123, 127, 129, 131, 133, 135, 137, 138, 140, 142, 144, 146, 148, 150, 153, 155, 157, 158, 160, 162, 164, 166, 168, 172, 174, 176, 178, 180, 182, 184, 186, 189, 191, 193, 195, 197, 199, 201, 203, 205, 207, 209, 211, 213, 215, 217, 219, 221, 223, 225, 227, 229, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412 and 414.

25. An isolated antibody as in claim 24 wherein the nucleic acid is encoded by a sequence selected from the group consisting of SEQ ID NO:'s 73, 184, 186 and 242.

26. An isolated antibody as in claim 24 which further comprises a toxin.

27. A method for detecting a tumor cell which comprises detecting the expression in said cell of a tumor-associated marker, wherein said marker is a nucleic acid selected from the group of nucleic acids in claim 24.

28. A method as in claim 27 wherein the nucleic acid marker is selected from the group consisting of SEQ ID NO:'s 73, 184, 186 and 242.

29. A method for detecting a tumor cell which comprises detecting the expression in said cell of a tumor-associated marker, wherein said marker is a polypeptide selected from the group consisting of SEQ ID NO:'s 10, 12, 14, 16, 20, 24, 46, 28, 30, 34, 36, 38, 40, 42, 44, 46, 48, 50, 52, 54, 56, 58, 60, 62, 64, 66, 68, 70, 71, 72, 74, 76, 78, 80, 82, 84, 86, 88, 90, 92, 94, 96, 98, 100, 102, 104, 106, 108, 110, 112, 114, 116, 118, 120, 124, 126, 128, 130, 132, 134, 136, 139, 141, 143, 145, 147, 149, 151, 152, 154, 156, 159, 161, 163, 165, 167, 169, 171, 173, 175, 177, 179, 181, 183, 185, 187, 188, 190, 192, 194, 196, 198, 200, 202, 204, 206, 208, 210, 212, 214, 216, 218, 220, 222, 224, 226, 228, 231, 233, 235, 237, 239, 241, 243, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 379, 381, 383, 385, 387, 389, 391, 393, 397, 399, 401, 403, 405, 407, 409, 411, 413 and 415.

30. A method as in claim 29 wherein the polypeptide marker is selected from the group consisting of sequence selected from the group consisting of SEQ ID NO:'s 74, 185, 187, 188 and 243.

31. A method for regulating the growth of a tumor cell which comprises altering the level of expression of a tumor-associated marker, wherein said marker is a nucleic acid selected from the group of nucleic acids of claim 24.

32. A method as in claim 31 wherein the nucleic acid marker is selected from the group consisting of sequences selected from the. group consisting of SEQ ID NO:'s 73, 184, 186 and 242.

33. A method as in claim 31 wherein the level of expression of the tumor-associated marker is regulated with an siRNA.

34. A method for regulating the growth of a tumor cell which comprises altering the level of expression of a tumor marker, wherein said marker is a polypeptide selected from the group of polypeptides of claim 29.

35. A method as in claim 34 wherein the polypeptide is selected from the group consisting of sequence selected from the group consisting of SEQ ID NO:'s 74, 185, 187, 188 and 243.

36. A method for preventing the growth of a tumor cell which comprises treating the cell with an antibody specific for a tumor-associated antigen wherein the antigen comprises a polypeptide as in claim 29.

37. A method as in claim 34 wherein the tumor marker is a polypeptide selected from the polypeptides of SEQ ID NO:'s 74, 185, 187, 188 and 242.

38. A method as in claims 36 or 37 wherein said antibody further comprises a toxin.

39. An isolated polypeptide for use as an immunogen, wherein said polypeptide is selected from the group of polypeptides of claim 29.

39. The isolated peptide of claim 37 or 38 which comprises an epitope reactive with a Cytotoxic T-cell.

40. A method for determining whether a nucleic acid is a marker for a stress-induced phenotype in a species which comprises:
(a) providing a database of expressed sequence tag sequences (EST's) from the species;
(b) placing said EST's in groups termed clusters based on homology of EST's within each cluster;
(c) determining for each cluster the total number of EST's within said cluster;
(d) ordering said clusters sequentially based on the number of EST's in each cluster;
(e) dividing said ordered clusters into subranges based on the number of EST's per cluster;
(f) determining for each cluster subrange obtained from step (e) the number EST's within said cluster which are expressed in a cell under said stress conditions;
(g) calculating according to a normal distribution the number of clusters in each subrange expected to contain a predetermined threshold percentage of EST's expressed in a cell under said stress conditions, wherein said threshold percentage is a percentage from about 10% to about 80%;
(h) determining the number of clusters in each subrange observed to contain said predetermined threshold percentage of EST's expressed in said cell; and
(i) identifying subranges having an observed number of clusters that meet said predetermined threshold percentage greater than the number of clusters expected to meet said predetermined threshold percentage for the subrange according to normal distribution;
wherein if the percentage of EST's expressed in said cell type of interest in a cluster identified in (i) is equal to or greater than said predetermined threshold percentage, said cluster contains a nucleic acid marker that is a marker for the stress-induced phenotype.

41. The method of claim 40 wherein one or more of the steps are performed on a computer.

42. The method of claim 40 wherein the individual clusters are divided into subranges exponentially.

43. The method of claim 40 wherein the individual clusters are divided into subranges linearly.

44. The method of claim 40 wherein the predetermined threshold percentage of EST's expressed in said cell type of interest is a percentage of about 80%.

45. The method of claim 40 wherein the species is Arabdopsis.

46. The method of claims 40 or 45 wherein the stress-induced phenotype is selected from the group consisting of hyperosmotic stress and high salt conditions.

47. A method for determining whether a nucleic acid is a marker for a tumor cell from a human which comprises:
(a) providing a database of expressed sequence tag sequences (EST's) from human tumor cells and human normal cells;
(b) placing said EST's in groups termed clusters based on homology of EST's within each cluster;
(c) determining for each cluster the total number of EST's within said cluster;
(d) ordering said clusters sequentially based on the number of EST's in each cluster;
(e) dividing said ordered clusters into subranges based on the number of EST's per cluster;
(f) determining for each cluster subrange obtained from step (e) the number EST's within said cluster which are expressed in a tumor cell;
(g) calculating according to a normal distribution the number of clusters in each subrange expected to contain a predetermined threshold percentage of EST's expressed in said human tumor cells, wherein said threshold percentage is a percentage from about 10% to about 100%;
(h) determining the number of clusters in each subrange observed to contain said predetermined threshold percentage of EST's expressed in a tumor cell; and
(i) identifying subranges having an observed number of clusters that meet said predetermined threshold percentage greater than the number of clusters expected to meet said predetermined threshold percentage for the subrange according to normal distribution;
wherein if the percentage of EST's expressed in said cell type of interest in a cluster identified in (i) is equal to or greater than said predetermined threshold percentage, said cluster contains a nucleic acid that is a marker for a tumor cell.

48. The method of claim 47 wherein one or more of the steps are performed on a computer.

49. The method of claim 47 wherein the individual clusters are divided into subranges exponentially.

50. The method of claim 47 wherein the individual clusters are divided into subranges linearly.

51. The method of claim 47 wherein the predetermined threshold percentage of EST's expressed in said cell type of interest is a percentage of about 80% to 100%.

52. The method of claim 47 wherein the predetermined threshold percentage of EST's expressed in said cell type of interest is a percentage of at least 90%.

53. The method of claim 47 wherein the predetermined threshold percentage of EST's expressed in said cell type of interest is a percentage of 100%.

54. The method of claim 47 wherein step (i) comprises identifying subranges having an observed number of clusters meeting said predetermined threshold percentage at least five times greater than the number expected for the subrange according to normal distribution.

55. The method of claim 47 wherein step h consists of (i) identifying subranges having an observed number of clusters meeting said predetermined threshold percentage at least one standard deviation greater than the number expected for the subrange according to normal distribution.
